(19) United States
(12) Patent Application Publication
(10) Pub. No.: US 2005/0216005 A1
(43) Pub. Date: Sep. 29, 2005

(54) Selective axis anchor screw posterior lumbar plating system

(76) Inventor: Robert S. Howland, Seal Beach, CA (US)

Correspondence Address: ORRICK, HERRINGTON & SUTCLIFFE, LLP;IP PROSECUTION DEPARTMENT, 4 PARK PLAZA, SUITE 1600, IRVINE, CA, 92614-2558, US

(21) Appl. No.: 11/133,708

(22) Filed: May 19, 2005

Related U.S. Application Data
(63) Continuation-in-part of application No. 10/889,754, filed on Jul. 12, 2004.
Application No. 10/889,754 is a continuation-in-part of application No. 10/698,087, filed on Oct. 31, 2003.
Application No. 10/698,087 is a continuation of application No. 10/133,310, filed on Apr. 24, 2002, now Pat. No. 6,770,075.
Application No. 10/133,310 is a continuation-in-part of application No. 09/861,278, filed on May 17, 2001, now Pat. No. 6,478,798.

(51) Int. Cl. A61B 17/58

(52) U.S. Cl. 606/61

(57) ABSTRACT

An anchor screw assembly includes a screw having a threaded portion and a head portion to which a swing bolt is pivotally coupled. A clamp assembly includes lower and upper clamp portions assembled into connecting beams that are securable on the swing bolt by a fastener. The clamp portions include first passages for receiving an intermediate region of the swing bolt therethrough to receivably retain the clamp assembly on the connecting beam and on the swing bolt. The head portion of the screw includes a shoulder, and the lower clamp portion has a seat that frictionally engages the shoulder when the clamp assembly is fully secured on the connecting beam and swing bolt, thereby securing the swing bolt relative to the screw. Multiple screw assemblies are screwed into adjacent vertebrae, and a connecting member is secured by the clamp assemblies between the anchor screw assemblies to stabilize the vertebrae. An optional cross-brace member interconnects two or more connecting members.

### RELATED APPLICATION INFORMATION

This application is a continuation-in-part of U.S. patent application Ser. No. 10/889,754, filed Jul. 12, 2004, which application is a continuation-in-part of U.S. patent application Ser. No. 10/698,087, filed Oct. 31, 2003, which is a continuation of U.S. patent application Ser. No. 10/133,310, filed Apr. 24, 2002, which is a continuation in part of U.S. patent application Ser. No. 09/861,278, filed May 17, 2001.

Each of the foregoing patent applications is hereby incorporated by reference in its entirety as if fully set forth herein.

### FIELD OF THE INVENTION

The present invention relates generally to apparatus and methods for treating spinal disorders, and more particularly to anchor screw assemblies, spinal fixation systems including such anchor screw assembles, and methods for stabilizing, adjusting, or otherwise fixing adjacent vertebrae using such spinal fixation systems.

### BACKGROUND

Various systems and methods have been suggested for treating spinal disorders, such as degenerative discs, stenosis, trauma, scoliosis, kyphosis, or spondylolisthesis. For example, U.S. Pat. No. 5,545,166, naming the same inventor as the present application, discloses a spinal fixation system that includes a plurality of anchor screws, clamp assemblies, pivot blocks, clamp blocks, and rods that are implanted along a patient's spine to fix two or more adjacent vertebrae relative to one another. The system generally includes a swing bolt anchor screw, a pivot block receivable on the swing bolt, and a clamp block receiving a rod therethrough that is pivotally attachable to the pivot block. In addition, the system includes one or more fixed anchor screws, and clamp assemblies for receiving the rod therein. The clamp assemblies and pivot block are receivable on the anchor screws by spindles that thread along a threaded portion of the anchor screws.

During use, vertebrae to be treated are surgically exposed, and an arrangement of anchor screws and clamp accessories are selected. For example, a fixed anchor screw may be screwed into each of the vertebrae on either side of a first vertebra. A rod is selected that may extend between the fixed anchor screws and that may be bent to conform to the shape of the anatomy encountered. The rod is inserted through a loose clamp block, and the rod is placed in clamp assemblies that are received over the fixed anchor screws.

A swing bolt anchor screw is then screwed into the first vertebra adjacent the rod, and a pivot block is received on the swing bolt screw. The clamp block and/or pivot block are adjusted such that the clamp block may be engaged with a pivot on the pivot block. A set screw may then be screwed into the clamp block to secure the clamp block to the pivot. A pair of set screws are also screwed into the clamp block to secure the rod within the clamp block. Preferably, a pair of such systems are implanted on either side of the vertebrae.

During the procedure, it may be desirable to adjust the vertebrae relative to one another. Once the system(s) is(are) connected as described above, the set screws may be loosened and the rod(s), clamp block(s), and/or pivot block(s) may be adjusted, e.g., by moving the spindle(s) to adjust the height of the pivot block(s) and/or clamp assemblies on the anchor screws, by pivoting the swing bolt anchor screw(s), and/or pivoting the clamp block(s) relative to the pivot block(s). Once the vertebrae have been moved into a desired position, the set screws may be tightened, and the spindles secured in position by crimping the walls surrounding the spindles.

An advantage of this system is that the swing bolt anchor screw, pivot block, and clamp block arrangement allows adjustment of the system about two axes, i.e., the axis of the swing bolt anchor screw and the axis of the pivot on the pivot block. However, because the system of the '166 patent is polyaxial, i.e., may pivot about multiple axes, there is greater risk of the system coming out of alignment when the patient resumes normal physical activity.

This system is also very complicated, involving six parts, including three set screws, that are mounted on each swing bolt anchor screw. In addition, because the swing bolt is threaded, an intricate spindle device is required in order to allow the pivot block and clamp assemblies to be threaded onto the swing bolt, and still control their orientation about the axis of the swing bolt. Thus, because of its complexity and many intricate parts, this system may be expensive to manufacture and/or difficult to implant.

Accordingly, apparatus and methods for stabilizing, adjusting, and/or fixing vertebrae would be considered useful.

### SUMMARY OF THE INVENTION

The present invention is directed to anchor screw assemblies, spinal fixation systems including such anchor screw assembles, and methods for stabilizing, adjusting, or otherwise fixing adjacent vertebrae using such spinal fixation systems.

In accordance with one aspect of the present invention, an anchor screw assembly is provided that includes a screw having a first threaded portion, and a second head portion. A swing bolt is pivotally coupled to the second portion of the screw, and may include a threaded region on its end opposite the screw. In addition, the swing bolt defines a first axis. In one embodiment, the swing bolt includes a circular region extending along the first axis, the circular region having a circular cross-section and a substantially smooth wall.

A clamp assembly is provided that includes first and second clamp portions that are receivable on the swing bolt and that are adapted to slidably engage a connecting beam, as described below. Each clamp portion has a first passage for receiving the swing bolt therethrough. In one embodiment, each clamp portion has a circular first passage for receiving the circular region of the swing bolt. Thus, the circular region of the swing bolt and the first passages each have circular cross-sections, thereby allowing rotation of the clamp assembly with respect to the swing bolt about the first axis.

In addition, the first and second clamp portions each have a cooperating mating portion therein, the cooperating mating portions are mated when they are brought together. When the clamp portions are so mated, the external surface of the clamp assembly forms a smooth cylindrical waist. The cylindrical waist is adapted to slide within a slot provided on a connecting beam or other structure to facilitate a selective axis feature described more fully below.

A fastener is also provided for securing the clamp assembly on the swing bolt and to thereby attach a connecting beam to the swing bolt. Preferably, the fastener is a jam nut, such as a twelve (12) point jam nut or a hex jam nut, that may be threaded onto the threaded region of the swing bolt to secure the clamp assembly, and connecting beam, onto the swing bolt.

In a preferred embodiment, the second portion of the anchor screw includes a shoulder, and the clamp assembly may substantially engage the shoulder when the clamp assembly and connecting beam are fully secured on the swing bolt, thereby preventing the swing bolt from pivoting with respect to the anchor screw. More preferably, the shoulder is provided with a profile about a pivot point on the second portion, and the lower clamp portion includes a recess adjacent its lower surface that intersects the first passage and that has a profile that matches the profile of the shoulder on the anchor screw. For example, the recess may have a matching spherical shape for slidably receiving a spherical shoulder therein as the clamp assembly pivots about the pivot point, i.e., before the clamp assembly is fully secured on the swing bolt. Alternatively, the recess may have a matching conical shape for receiving a conically shaped shoulder therein.

In accordance with another aspect of the present invention, a spinal fixation system is provided that includes at least two anchor screw assemblies, such as those described above. Each of the anchor screw assemblies includes an anchor screw having a threaded portion, and a swing bolt pivotally coupled to the anchor screw. Each anchor screw assembly also includes a clamp assembly having a passage for receiving the swing bolt therethrough, and a fastener for securing the clamp assembly to the swing bolt and to a connecting beam.

The spinal fixation system also includes one or more connecting beams extending between and structurally interconnecting the anchor screw assemblies. The connecting beam is preferably a generally flat, elongated member having a width that is substantially larger than its height, thereby creating an elongated “plate” shape. Alternatively, the connecting beam may include two or more generally flat, elongated segments. In one embodiment, the connecting beam is a one-level connecting beam interconnecting two anchor screw assemblies, including a single connecting beam having an elongated slot on each end thereof. One anchor screw assembly is attached to each end of the one-level connecting beam by having the swing bolt pass through the clamp assembly, which is assembled into the elongated slot, with the second clamp portion of the clamp assembly engaging the bottom surface of the connecting beam at the slot and the first clamp portion engaging the top surface of the connecting beam at the slot, and with the waist portion of the clamp assembly slidably retained within the slot.

In another embodiment, the connecting beam is a multi-level connecting beam interconnecting three or more anchor screw assemblies. For example, a two-level connecting beam may be used to interconnect three anchor screw assemblies, including a substantially rigid connecting beam having elongated slots on each end and a third elongated slot near the center thereof. One anchor screw assembly is attached to each end and the third anchor screw assembly is connected to the center of the two-level connecting beam by having the swing bolt pass through the clamp assembly, which is assembled into the elongated slot, with the waist portion of the clamp assembly slidably retained within the slot.

In another embodiment, a single or multi-level connecting beam interconnecting two or more anchor screw assemblies may include a plurality of integrated segments in a pre-formed connecting beam construction, with each segment extending in a plane that forms a connecting angle with the plane of the adjacent segment. The connecting angle is preferably an acute angle within the range of 0° to about 25°. The pre-formed connecting beam may be adapted to interconnect two, three, or more anchor screw assemblies. Advantageously, the overall length and size of the pre-formed connecting beam, the length and size of each segment, the connecting angles, and other features of the pre-formed connecting beam may be selected to accommodate the anatomy of the patient for desired results.

In another embodiment of a multi-level connecting beam system, the connecting beam includes a three-piece hinged beam assembly. The three-piece hinged beam assembly includes two beam side-sections and a beam center-section. Each of the beam side-sections is connected to the center-section by a hinge, thereby allowing the beam side-sections to pivot relative to the center-section. Each of the side-sections also includes an elongated slot near its end opposite the center-section. The center-section also includes an elongated slot, which is preferably offset by 90° relative to the elongated slots on the beam side-sections. The three-piece hinged beam assembly is connected to the three anchor screw assemblies by having the swing bolt of each of the three anchor screw assemblies pass through one of the clamp assemblies assembled into the elongated slots of the three-piece hinged beam assembly, with the second clamp portion engaging the bottom surface of the connecting beam at the slot and the first clamp portion engaging the top surface of the connecting beam at the slot, and with the waist portion of the clamp assembly slidably retained within the slot.

In accordance with yet another aspect of the present invention, a spinal fixation system is provided that includes a plurality of anchor screw assemblies, clamp assemblies, fasteners, and connecting beams, such as those described above. The spinal fixation system also includes one or more cross-brace members extending between and interconnecting two or more connecting beams. The cross-brace members are preferably elongated members having sufficient structural rigidity to perform the function of bracing two adjacent spinal fixation systems to which the cross-bracing is connected. A connection mechanism is provided to connect each end of the cross-brace member to a respective connecting beam. The connection mechanism may comprise a stud formed on one or the other of the cross-brace member or the connecting beam, and a mating hole or slot formed on the other member. A fastener may be used to mate the members together. Preferably, the cross-brace members are formed from materials the same as or similar to those used in the remainder of the spinal fixation system. The cross-brace members may be used with any of the different forms of connecting beams described herein, or other connecting beams known in the art.

One advantage provided by the spinal fixation systems described herein is the provision of a selective axis feature whereby the clamp assembly engages the connecting beam at a substantially perpendicular angle. In particular, on assembly, the spherical shoulder of the anchor screw loads against the spherical seat of the lower clamp. The swing bolt of the anchor screw assembly passes up through the central passage formed by the clamp assembly. The fastener is threaded onto the end of the swing bolt down to the flat upper face of the upper clamp. On final assembly, the fastener and the spherical shoulder of the anchor screw tighten the clamp assembly firmly against the connecting beam at 90° force. Prior to final tightening of the fastener, the clamp assembly slides in the connecting beam slot to locate the swing bolt centerline, after which the fastener is tightened at approximately 120-140 inch pounds of force.

In accordance with another aspect of the present invention, a method is provided for simple alignment or otherwise stabilizing vertebrae relative to one another using a plurality of swing bolt anchor screw assemblies, such as those described above. A threaded portion of a first swing bolt anchor screw is screwed into a first vertebra until a first pivot axis of the first swing bolt anchor screw is generally parallel to the spinal axis. A threaded portion of a second swing bolt anchor screw is screwed into a second vertebra adjacent the first vertebra until a second pivot axis of the second swing bolt anchor screw is either generally parallel to the spinal axis, or substantially transverse to the first pivot axis, depending on the type and orientation of connecting beam being used. If desired, a third anchor screw (or more) may be screwed into other vertebra adjacent to the first vertebra. An angle of one or more swing bolts on the first and second swing bolt anchor screws may be adjusted about the first and second pivot axes.

An appropriate number of clamp assemblies are then assembled within the slots of a connecting beam. The clamp assemblies are assembled onto the connecting beam by placing an upper clamp portion through the top of each slot and placing a lower clamp portion through the bottom of each slot, thereby mating each pair of clamp portions to form an assembled clamp assembly. Each clamp assembly preferably forms a narrowed waist portion that slides within its respective elongated slot on the connecting beam.

The connecting beam assembly may be placed on the swing bolts of the first and second swing bolt anchor screws, either before or after the angle adjustments described above. This is accomplished by directing each of the clamp assemblies over its respective swing bolt, such that the swing bolt passes through the passage formed by the clamp assembly. A seat portion on the lower-facing surface of each of the lower clamp portions engages the upper shoulder surface of the respective anchor screw. In a preferred embodiment, the upper shoulder surface is spherical, and mates with a spherical seat portion on the lower-facing surface of each lower clamp portion. The connecting beam may be a single level connecting beam, or a multi-level connecting beam, as described above. The swing bolt of each anchor screw is directed through the clamp assembly located in the respective slot in the connecting beam.

Finally, a fastener is then attached to the upper threaded portion of each of the swing bolts. For example, a nut or other fastener may be threaded onto the swing bolt after the clamp assemblies are positioned, thereby securing the connecting beam between the upper and lower clamp portions and securing the clamp assemblies on the connecting beam and swing bolts. Prior to tightening the fasteners, the swing bolt and clamp assembly slides in the slot to locate the vertical centerline of the swing bolt. These fasteners may also be loosened to allow adjustment of the vertebrae relative to one another, and then the fasteners may again be tightened to fix the vertebrae in desired relative positions.

Other objects and features of the present invention will become apparent from consideration of the following description taken in conjunction with the accompanying drawings.

### BRIEF DESCRIPTION OF THE DRAWINGS

FIG. 1 is a perspective view of a preferred embodiment of a multi-level selective axis spinal plating fixation system as implanted between vertebrae of a patient, in accordance with the present invention.

FIG. 2 is another perspective view of the multi-level selective axis spinal plating fixation system of FIG. 1.

FIG. 3 is a perspective view of a preferred embodiment of a multi-level selective axis spinal plating fixation system, in accordance with the present invention.

FIG. 4 is a perspective view of a preferred embodiment of a one-level selective axis spinal plating fixation system as implanted between vertebrae of a patient, in accordance with the present invention.

FIG. 5 is a perspective view of a preferred embodiment of a one-level selective axis spinal plating fixation system, in accordance with the present invention.

FIG. 6 is a perspective view of another preferred embodiment of a multi-level selective axis spinal plating fixation system, in accordance with the present invention.

FIG. 7 is a cross-sectional view of the upper end of an anchor screw assembly shown attached to a connecting beam.

FIGS. 8A-8C are first and second side views and a perspective view, respectively, of an assembled screw and swing bolt for an anchor screw assembly, in accordance with the present invention.

FIG. 8D is a cross-sectional view of the assembled screw and swing bolt shown in FIGS. 8A-8C, as taken along line A-A in FIG. 8B.

FIGS. 9A-9C are perspective, side, and top views, respectively, of an anchor screw, in accordance with the present invention.

FIGS. 10A-D are perspective, first and second side views, and a top view, respectively, of a swing bolt, in accordance with the present invention.

FIGS. 11A-C are perspective, side, and top views, respectively, of an embodiment of an upper clamp portion for a clamp assembly, in accordance with the present invention.

FIGS. 12A-C are perspective, side, and top views, respectively, of an embodiment of a lower clamp portion for a clamp assembly having a spherical seat, in accordance with the present invention.

FIGS. 13A-C are perspective, side, and top views, respectively, of an embodiment of a lower clamp portion for a clamp assembly having a conical or tapered seat, in accordance with the present invention.

FIGS. 14A-C are perspective, top, and side views, respectively, of a one-level connecting beam, in accordance with the present invention.

FIGS. 15A and 15B are top and side views, respectively, of a multi-level connecting beam, in accordance with the present invention.

FIGS. 16A-C are perspective, top, and side views, respectively, of a center-portion of the multi-level connecting beam shown in FIGS. 15A and 15B.

FIGS. 17A-C are perspective, top, and side views, respectively, of a side-portion of the multi-level connecting beam shown in FIGS. 15A and 15B.

FIGS. 18A-C are perspective, top, and side views, respectively, of a two-level connecting beam, in accordance with the present invention.

FIGS. 19A and 19B are top and side views, respectively, of a jam nut, in accordance with the present invention.

FIGS. 20A and 20B are first and second side views, in partial cross-section, of the upper end of an anchor screw assembly shown attached to a connecting beam.

FIGS. 21A-C are first and second side views and a perspective view, respectively, of an assembled screw and swing bolt for an anchor screw assembly, in accordance with the present invention.

FIG. 21D is a cross-sectional view of the assembled screw and swing bolt shown in FIGS. 21A-21C, as taken along line A-A in FIG. 21B.

FIGS. 22A-22C are perspective, side, and top views, respectively, of an anchor screw, in accordance with the present invention.

FIG. 22D is a side view of the upper end of the anchor screw shown in FIGS. 22A-C.

FIGS. 23A-D are perspective, first and second side views, and a top view, respectively, of a swing bolt, in accordance with the present invention.

FIGS. 24A-E are perspective, first top, first and second side, and second top views, respectively, of an embodiment of a lower clamp portion for a clamp assembly having a cylindrical seat, in accordance with the present invention.

FIG. 25 is a perspective view of a preferred embodiment of another multi-level selective axis spinal plating fixation system, in accordance with the present invention.

FIG. 26 is a perspective view of a multi-level pre-formed connecting beam, in accordance with the present invention.

FIG. 27 is a perspective view of a preferred embodiment of a pair of multi-level selective axis spinal plating fixation systems including a cross-brace, in accordance with the present invention.

FIG. 28 is a perspective view of a preferred embodiment of another pair of multi-level selective axis spinal plating fixation systems including a cross-brace, in accordance with the present invention.

FIG. 29 is a perspective view of a preferred embodiment of a pair of one-level selective axis spinal plating fixation systems including a cross-brace, in accordance with the present invention.

FIG. 30 is a perspective view of a multi-level pre-formed connecting beam.

FIG. 31 is a perspective view of another multi-level pre-formed connecting beam.

FIGS. 32A and 32B are top and side views, respectively, of a jam nut, in accordance with the present invention.

FIG. 33 is a side view, in partial cross-section, of the upper end of an anchor screw assembly shown attached to a connecting beam.

### DETAILED DESCRIPTION OF THE PREFERRED EMBODIMENTS

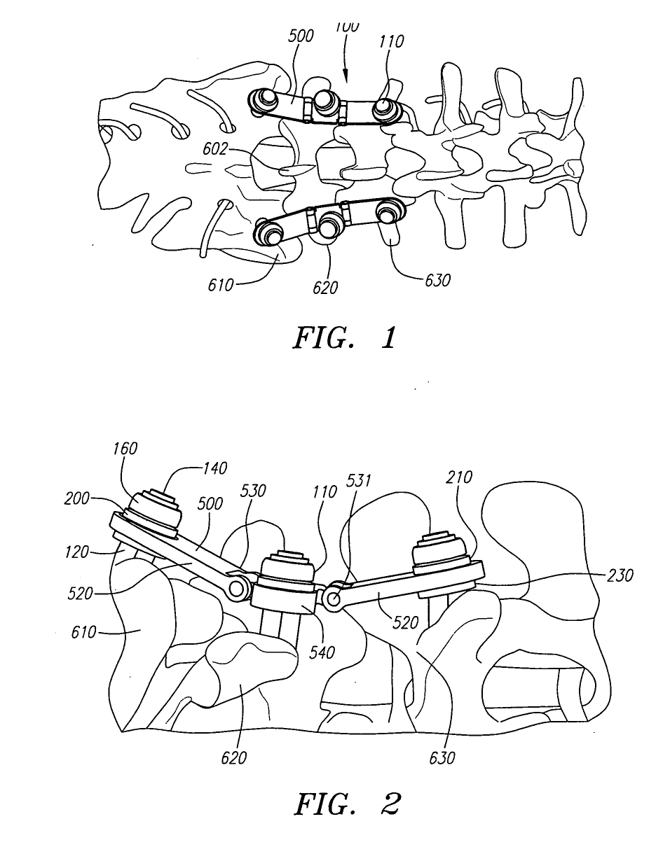

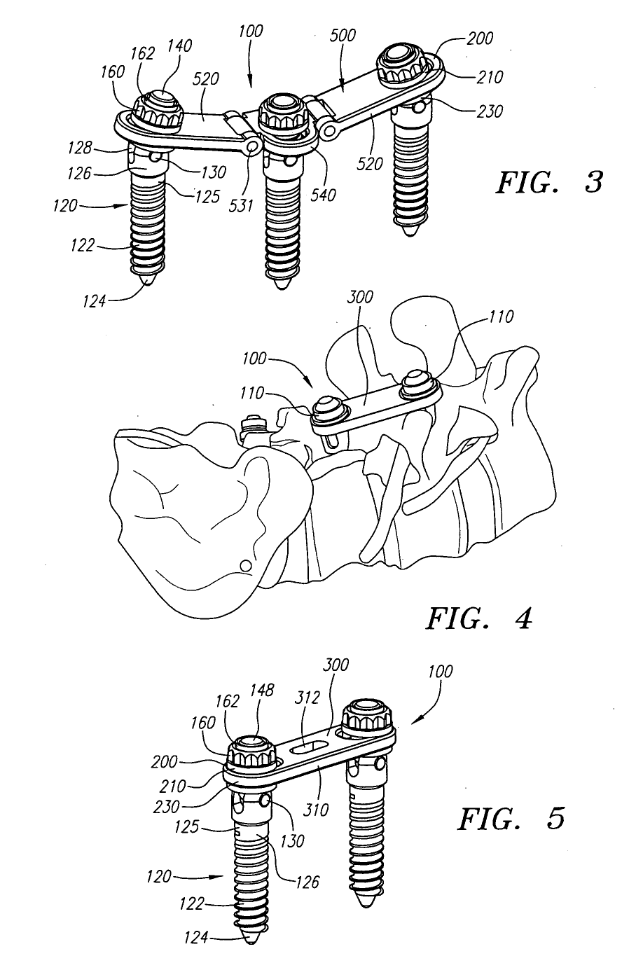

Turning now to the drawings, FIGS. 1-6 show preferred embodiments of a spinal fixation system 100, in accordance with the present invention. Generally, the spinal fixation system 100 includes a plurality of anchor screw assemblies 110 and a connecting beam, such as a one-level connecting beam 300, a two-level connecting beam 400, a multi-level connecting beam 500, or a pre-formed connecting beam 700. In FIGS. 1, 2, and 4, the spinal fixation systems 100 are shown as implanted between vertebrae of a patient. As shown there, an anchor screw 120 of each of the anchor screw assemblies 110 is screwed into one of several adjacent vertebrae 610, 620, 630. A connecting beam 300, 500 is attached to the upper end of each of the anchor screw assemblies, and extends between and interconnects the anchor screw assemblies. Because the connecting beams 300, 500 are substantially rigid, the spinal fixation system 100 is able to fix or stabilize the relative positions of the vertebrae to which the system is attached.

All of the components of the spinal fixation system 100 may be made from a variety of biocompatible materials, e.g., metals, and preferably from titanium or alloys including titanium.

Turning to FIGS. 3, 5, 6, 25, and 27-29, additional details relating to the structure of the spinal fixation system are shown. The spinal fixation system 100 includes two or more anchor screw assemblies 110, and a connecting beam 300, 400, 500, 700. The anchor screw assembly 110 includes an anchor screw 120, a swing bolt 140 that is secured by a pin 130 to the anchor screw 120, a clamp assembly 200 including an upper clamp portion 210 and a lower clamp portion 230 having a passage through which the swing bolt 140 extends, and a fastener 160 attached to the upper end of the swing bolt 140.

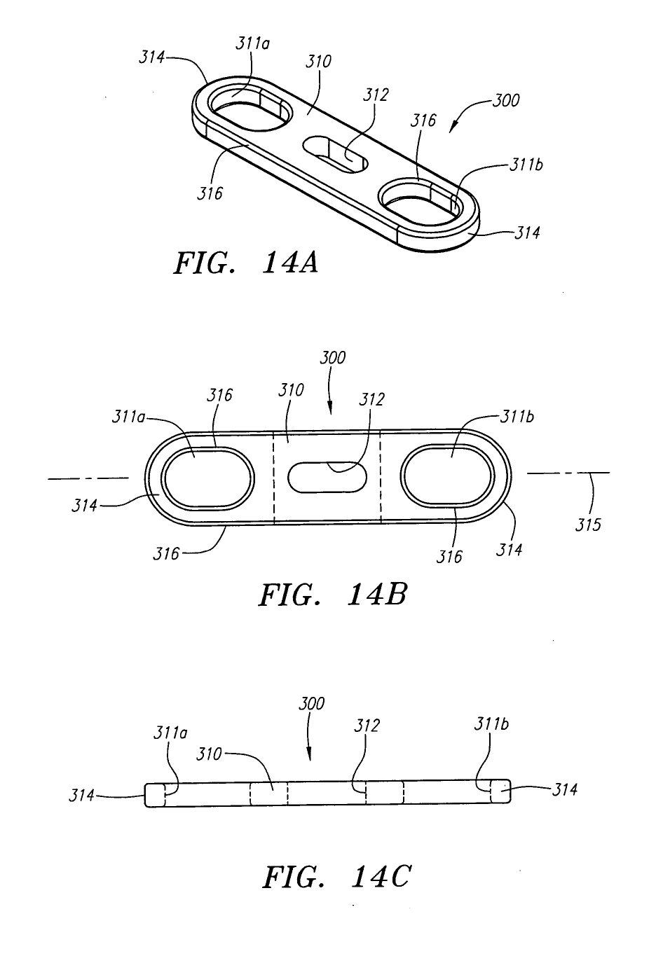

The one-level connecting beam 300 (FIG. 5) may be used with two anchor screw assemblies 110. The one-level connecting beam is preferably a generally flat, elongated member 310 having an elongated slot 311a, 311b (see FIGS. 14A-C) formed near each end for the purpose of engaging the clamp assembly 200 and the anchor screw assemblies 110 in a manner described more fully below. The connecting beam 300 is generally “plate”-shaped, meaning that it has a width that is substantially greater than its height. The same is generally true of the two-level connecting beams 400, the multi-level hinged connecting beams 500, and the pre-formed connecting beams 700 described below. The one-level connecting plate 300 also preferably includes a central aperture 312 for reducing the stiffness of the connecting beam and to allow bone graft and body fluid access.

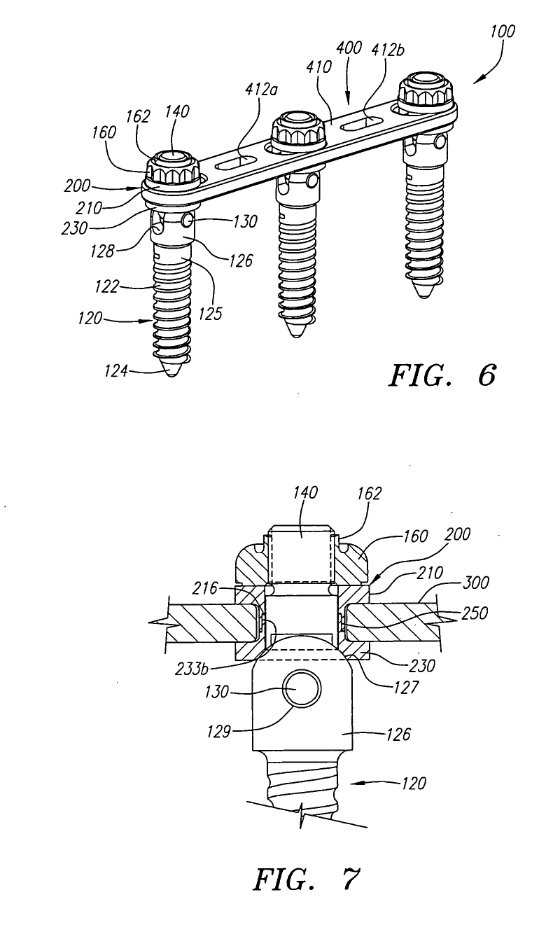

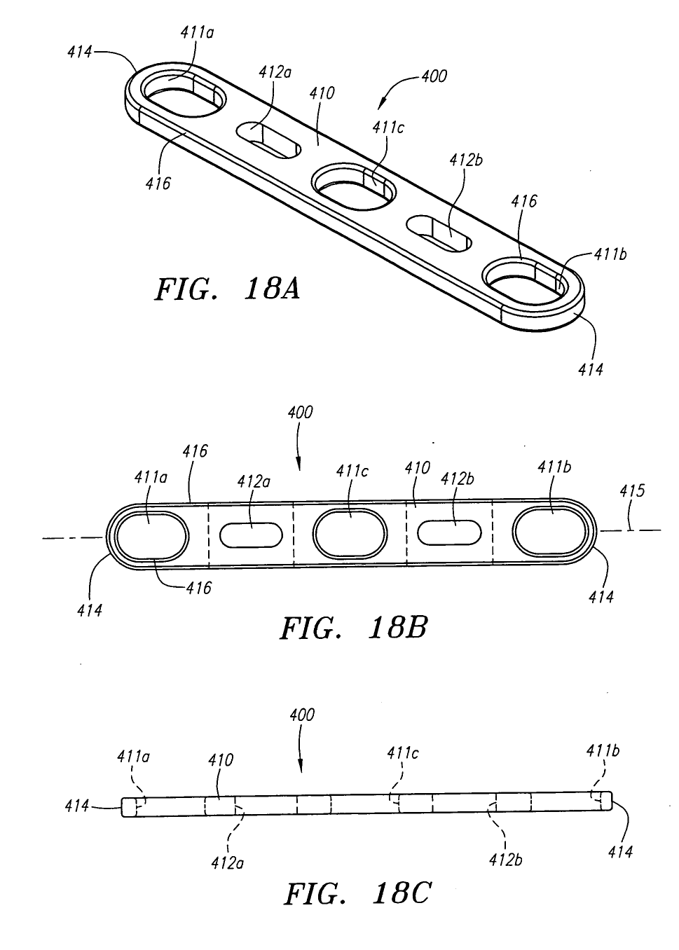

The two-level connecting beam 400 (FIG. 6) may be used with three anchor screw assemblies 110. The two-level connecting beam is also preferably a generally flat, elongated member 410 having an elongated slot 411a, 411b (see FIGS. 18A-C) formed near each end, and another elongated slot 411c (FIGS. 18A-C) formed near its center, all for the purpose of engaging the clamp assembly 200 and the anchor screw assemblies 110 in a manner described more fully below. The two-level connecting beam 400 also preferably includes a pair of central apertures 412a-b for reducing the stiffness of the connecting beam and to allow bone graft and body fluid access.

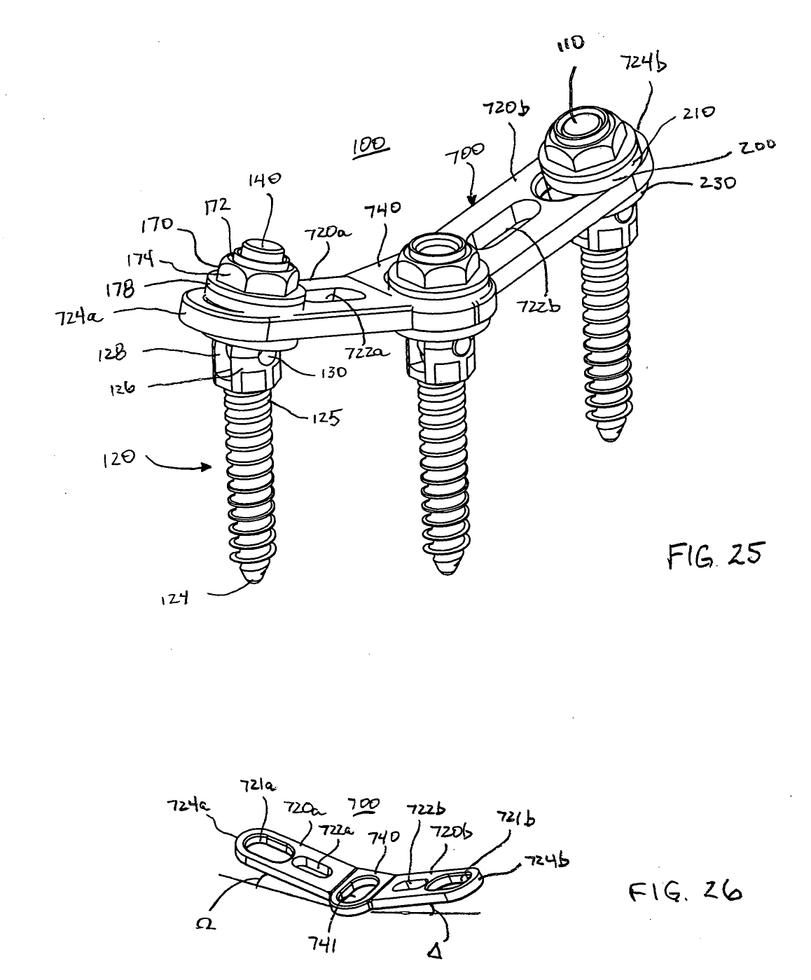

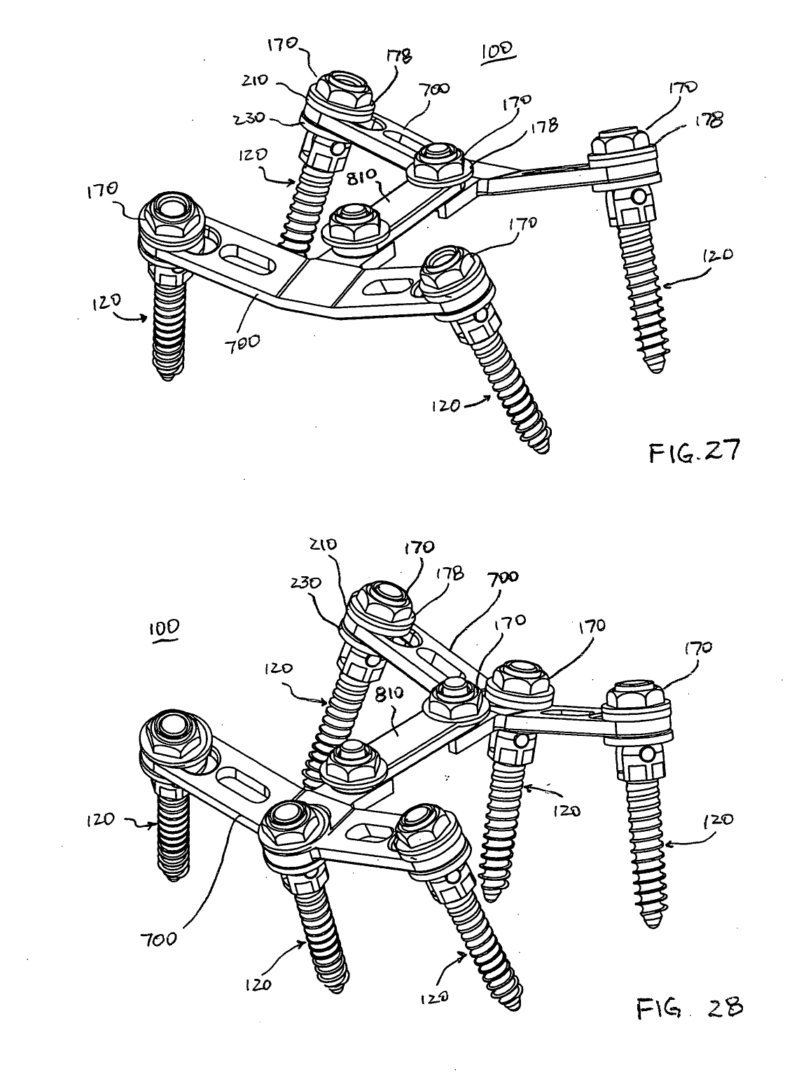

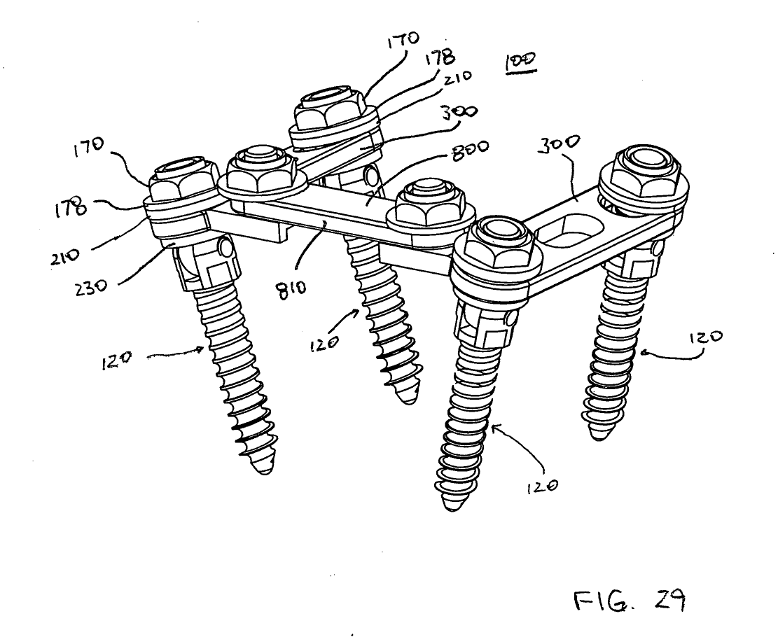

The pre-formed connecting beam 700 (FIGS. 25, 27, and 28) may be used with two or more anchor screw assemblies 110. The pre-formed connecting beam 700 includes a plurality of integrated segments, with each segment extending in a plane that forms a connecting angle with the plane of the adjacent segment. The connecting angle is preferably an acute angle in the range of from about 0° to about 25°, depending upon the clinical need. For example, the pre-formed connecting beam shown in FIGS. 25 and 26 includes three segments: a center segment and a pair of end segments. Each of the end segments includes an elongated slot formed near its end opposite the center segment for the purpose of engaging the clamp assembly 200 and the anchor screw assemblies 110. The center segment also includes an elongated slot, which is preferably offset by about 90° from the direction of elongation of the slots on the end segments. The center segment elongated slot is intended to engage the clamp assembly 200 and the third anchor assembly 110. The pre-formed connecting beam may be adapted to interconnect two, three, or more anchor screw assemblies. See, for example, the pre-formed connecting beam shown in FIG. 27, in which no slot is provided on the center segment of each connecting beam, and each connecting beam interconnects two anchor screw assemblies.

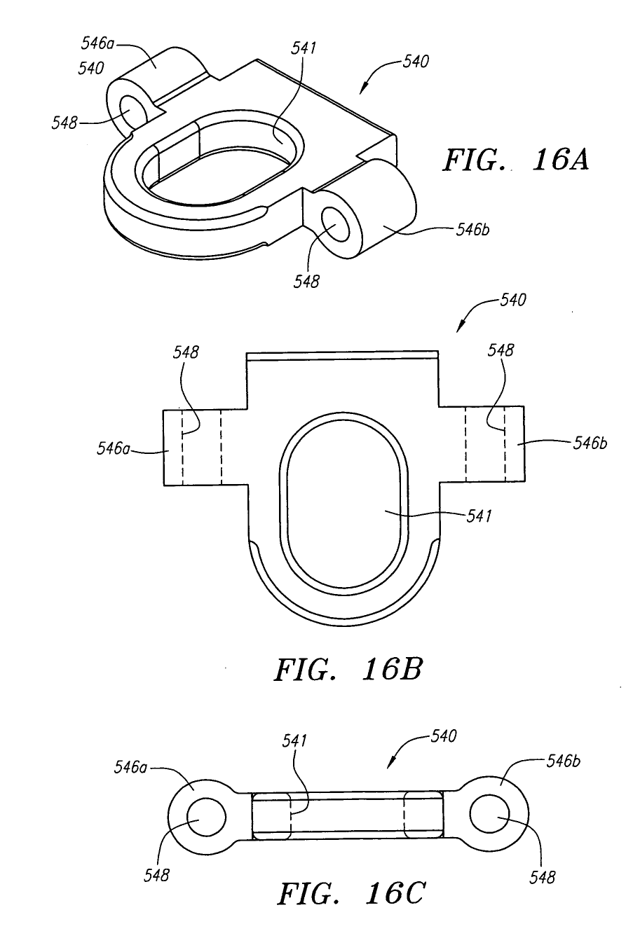

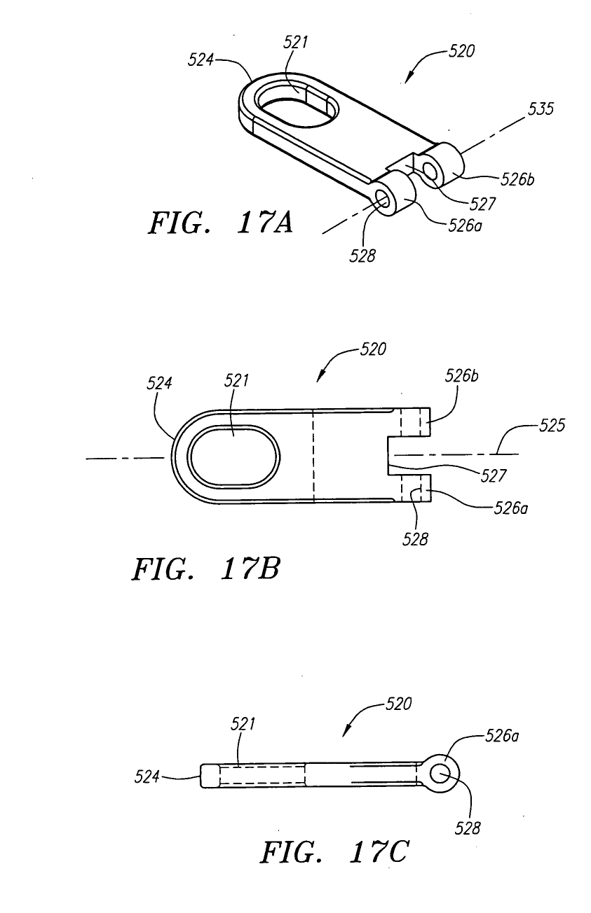

The multi-level hinged connecting beam 500 (FIG. 3) may also be used with three anchor screw assemblies 110. The multi-level connecting beam includes three primary components, a pair of side-portions 520 and a center-portion 540. Each of the side-portions 520 includes an elongated slot 521 (see FIGS. 17A-C) formed near its end opposite the center-portion for the purpose of engaging the clamp assembly 200 and the anchor screw assemblies 110. The center-portion 540 also includes an elongated slot 541 (see FIGS. 16A-C), which is preferably offset by about 90° from the direction of elongation of the slots 521 on the side-portions 520. The center-portion elongated slot 541 is intended to engage the clamp assembly 200 and the third anchor assembly 110. Each of the two side-portions 520 of the connecting beam is attached to an opposed side of the center-portion by a hinge 530 and hinge pin 531.

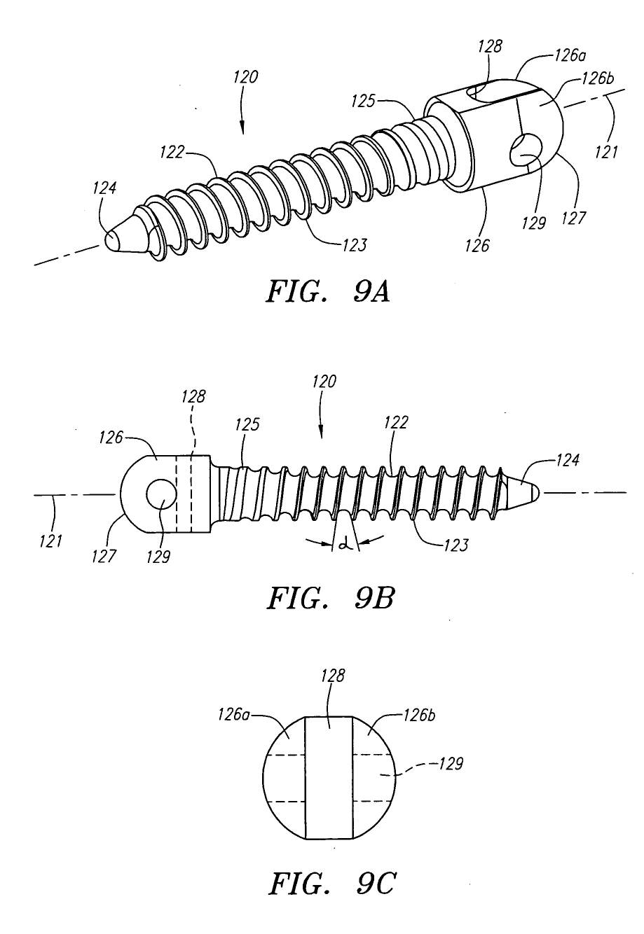

The component parts of the spinal fixation system 100 will now be described in more detail by reference to the drawings. Turning to FIGS. 9A-C, an anchor screw 120 is shown in additional detail. The screw 120 generally includes a first threaded portion 122 terminating in a tip 124, and a second head portion 126 opposite the tip 124. The threaded portion 122 may include a helical thread 123 defining a thread pattern, preferably configured for substantially securing the screw 120 into bone, such as a portion of a vertebra (not shown). The thread spacing may be between about three to six threads per centimeter (3-6 threads/cm), and preferably about 4.8 threads per centimeter (about 12 threads per inch). The thread spacing may be substantially constant between the tip 124 and the head portion 126 or may vary along the length of the threaded portion 122.

The leading and trailing edges of axially adjacent portions of the thread 123 may define an inclusive angle “alpha” between them of between about thirty to sixty degrees (30-60°), and preferably about forty-five degrees (45°). Preferably, each thread 123 tapers outwardly from the tangent of the root radius to the major diameter of the thread 123, such that the leading and trailing edges on either side of a portion of the thread 123 define tangent lines that intersect one another adjacent the outer edge of the respective portion of the thread 123. The thread 123 may have a height of between about 0.80-1.07 millimeters, and preferably between about 0.86-1.01 millimeters, with a root radius of about 0.84 millimeters.

The threaded portion 122 may have desired dimensions to accommodate threading into bone, such as a vertebra (not shown). For example, the threaded portion 122 may have an outer diameter between about 3.5-8.5 millimeters, preferably between about 5.6-8.6 millimeters, and a length between about 25-65 millimeters, and preferably between about 35-65 millimeters. The threaded portion 122 may have a substantially uniform major and minor diameter along its length. Alternatively, the threaded portion 122 may have a taper, e.g., increasing in minor diameter from the tip portion 124 towards the head portion 126. The thread 123 may have a substantially uniform height, or may become increasingly lower from the tip portion 124 towards the head portion 126, e.g., if the threaded root portion 122 is tapered, to provide a substantially uniform outer diameter for the threaded portion 122.

Preferably, the threaded portion 122 includes a pull-out portion 125. For example, the final two threads 125 before the head portion 126 may include a minor diameter that gradually expands out to the major diameter. In addition or alternatively, the final two threads 125 may have a plateau on their outer edge. This pull-out portion 125 may facilitate manufacturing of the anchor screw 120 and/or may improve engagement of the screw 120 with bone into which the screw 120 is threaded. Other thread patterns and screw designs that may be appropriate for use in an anchor screw assembly in accordance with the present invention may be found in U.S. Pat. Nos. 4,854,311, 5,034,011, and 5,226,766, the disclosures of which are expressly incorporated herein by reference.

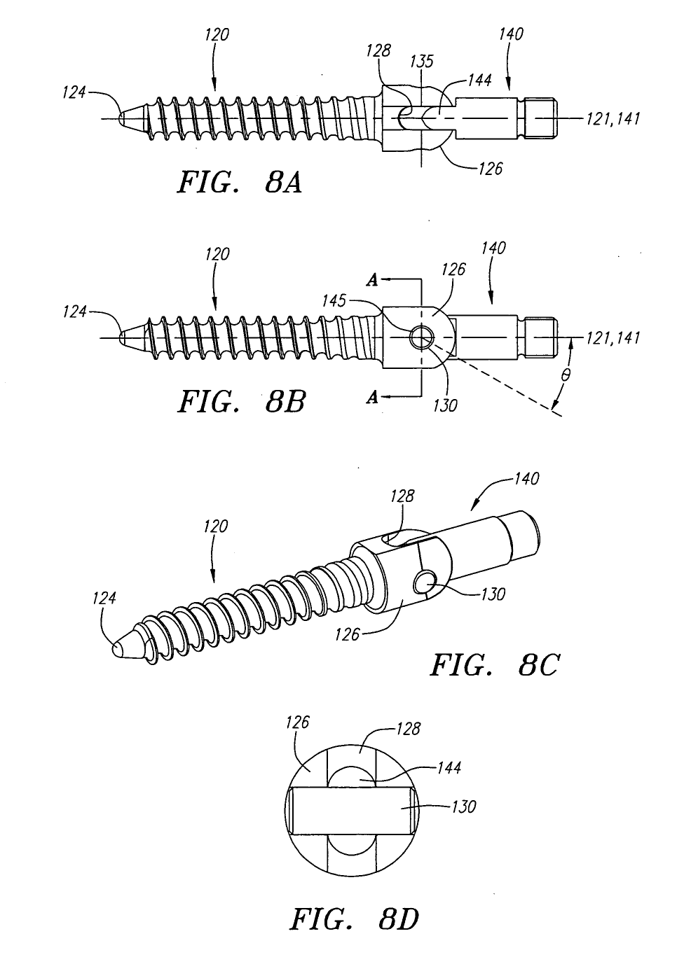

The head portion 126 generally has a cross-section larger than the threaded portion 122 and includes a spherical-radius shoulder 127 opposite the threaded portion 122. The shoulder 127 includes a predetermined spherical radius about a pivot axis 135 (see FIGS. 8A-D) to facilitate pivoting of the swing bolt 140 (see FIGS. 8A-D) with respect to the head portion 126, as explained further below. The head portion 126 includes a slot 128 therein extending generally parallel to a longitudinal axis 121 of the screw 120, thereby dividing the head portion 126 into ears 126a-b. Pin holes 129 extend through the ears 126a-b along the pivot axis 135, i.e., substantially perpendicular to the longitudinal axis 121.

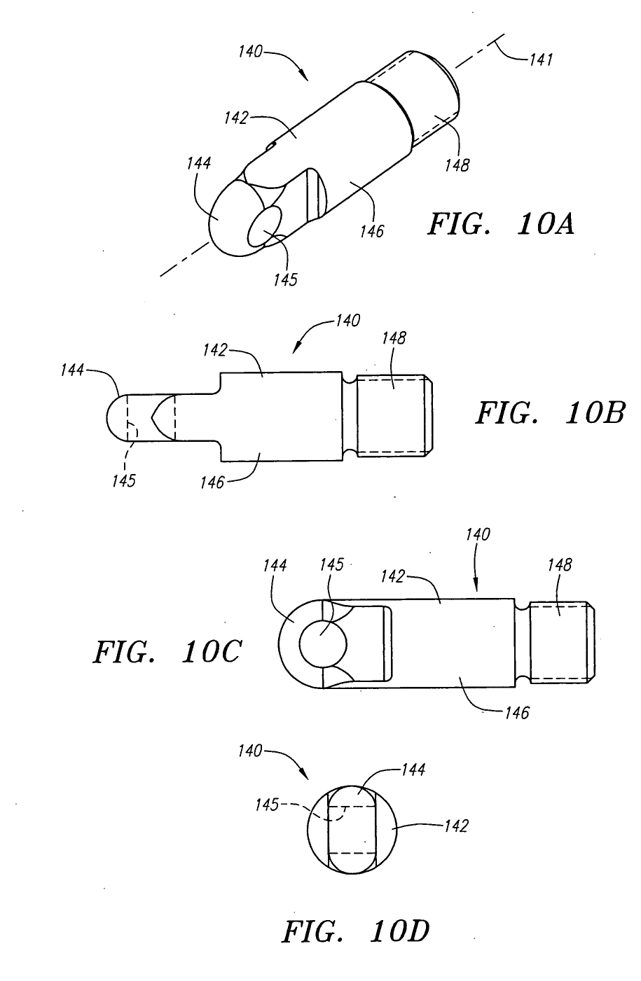

Turning to FIGS. 10A-10D, the swing bolt 140 includes an elongate body 142 including a first looped region 144, a second intermediate region 146, and a third threaded region 148 generally opposite the looped region 144. The looped region 144 may be substantially narrower than the other regions of the swing bolt 140, i.e., having a width slightly smaller than a width of the slot 128 in the screw 120 such that the looped region 144 may be received in the slot 128 between the ears 126a-b, as shown in FIGS. 8A-8D. The looped region 144 has a pin hole 145 therethrough that extends substantially perpendicular to the longitudinal axis 141.

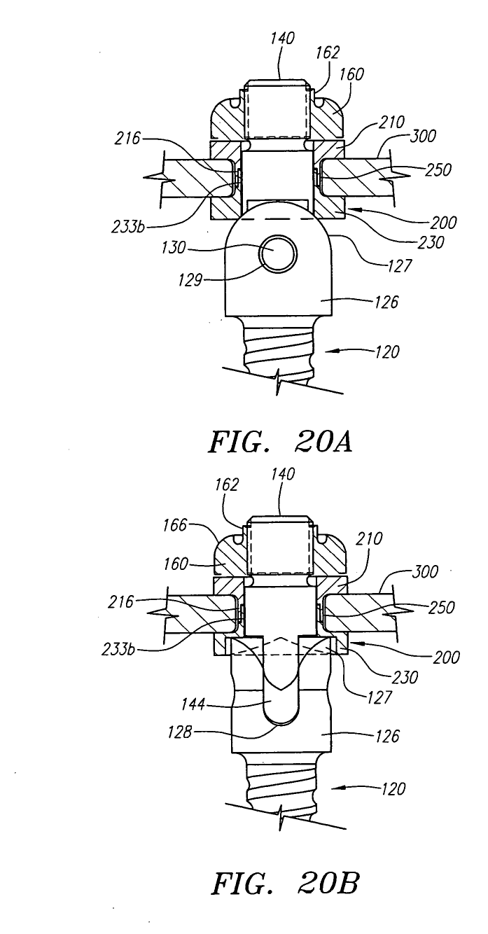

The intermediate region 146 of the swing bolt 140 is preferably substantially smooth-walled. In a first embodiment, shown in FIGS. 10A-D, the intermediate region 146 has a circular cross-section to form a generally cylindrical shape. This allows the clamp assembly 200 (see FIGS. 7, 20A, and 20B) to be slidably recieved on the intermediate region 146 while allowing full rotation of the clamp assembly 200 about the longitudinal axis 141.

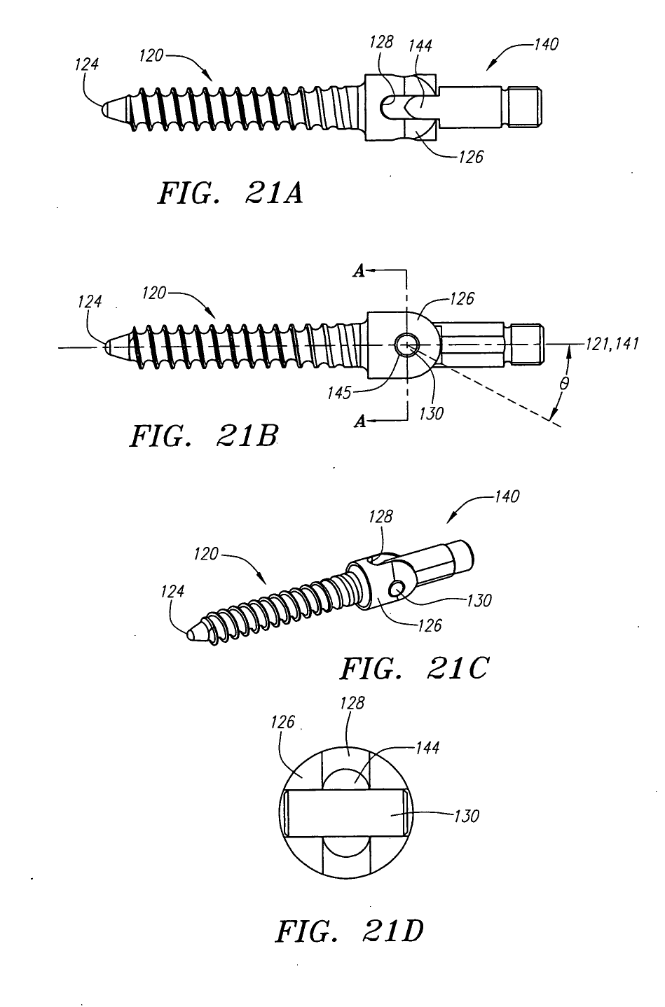

As shown in FIGS. 8A-8D, the looped region 144 of the swing bolt 140 is received in the slot 128 of the head portion 126 of the anchor screw 120, and a pin 130 is received through the pin holes 129, 145 to complete the anchor screw assembly. The pivot pin 130 assembles the swing bolt 140 to the screw 120, while allowing the swing bolt 140 and the screw 120 to pivot with respect to one another such that the longitudinal axes 121, 141 intersect, but define an angle “theta” (“θ”) greater than zero degrees, as shown in phantom in FIG. 8B. A similar structure is shown in FIGS. 21A-D for an alternative embodiment of the swing bolt 140.

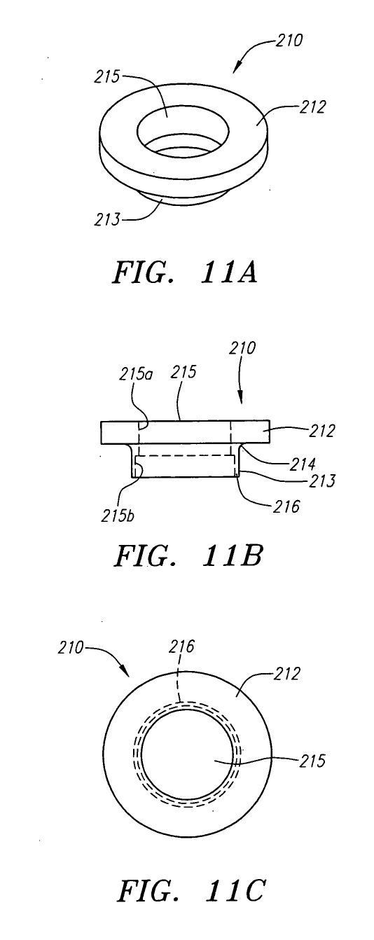

Turning to FIGS. 11A-C, 12A-C, and 13A-C, the components of the clamp assembly 200 (FIG. 7) are shown, including an upper clamp portion 210 and a lower clamp portion 230. The upper clamp portion 210 (see FIGS. 11A-C) is preferably an integrated body having a generally disc-shaped top portion 212 and a generally cylindrical bottom portion 213. A radius 214 is formed on the exterior of the upper clamp where the top portion 212 meets the bottom portion 213. A central passage 215 is formed through the upper clamp 210. The external width or diameter of the top portion 212 is variable, although it is greater than the width of the elongated slots contained on the connecting beams 300, 400, 500, in order to allow the clamp assembly 200 to engage the connecting beam. The external width or diameter of the bottom portion 213 is less than that of the top portion 212, such that the bottom portion 213 is able to fit slidably within the elongated slots provided on the connecting beams.

As noted above, the central passage 215 is a generally cylindrical conduit that passes through the top portion 212 and bottom portion 213 of the upper clamp 210. The central passage 215 has two sections, an upper section 215a and a lower section 215b. The upper section 215a of the central passage 215 has a diameter that is slightly larger than the diameter of the intermediate region 146 of the swing bolt 140, to provide a close fit with the intermediate region 146 when the upper clamp 210 is placed over the swing bolt 140. The lower section 215b of the central passage 215 has a slightly larger diameter than that of the upper section 215a, which slightly larger diameter is achieved by providing an area of thinning of the wall 216 forming the bottom portion 213 of the upper clamp 210. The slightly larger internal diameter of the lower section 215b of the central passage 215 is intended to accommodate a mating portion of the lower clamp portion 230 to provide a press fit between the upper clamp 210 and lower clamp 230 forming the clamp assembly 200 on assembly to a connecting beam, as described below and as shown, for example, in FIG. 7.

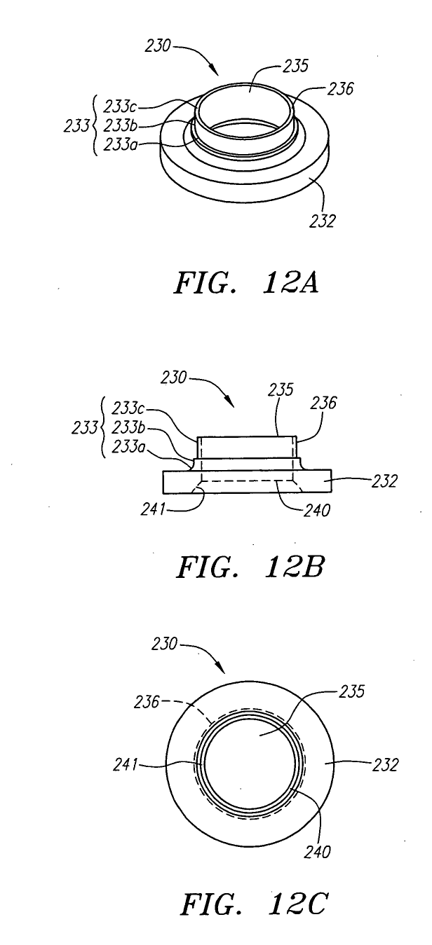

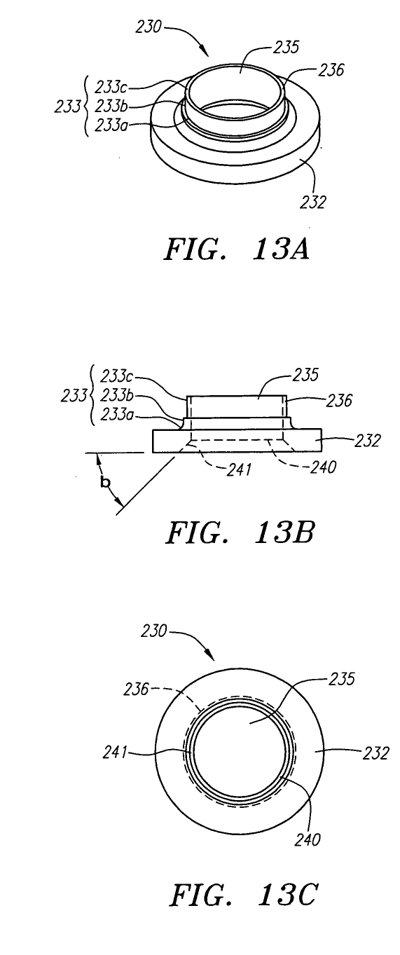

Turning to FIGS. 12A-C, 13A-C, and 24A-E, the lower clamp portion 230 is preferably an integrated body having a generally disc-shaped bottom portion 232 and a generally cylindrical top portion 233. The top portion 233 includes a radius portion 233a that extends from the top of the bottom portion 232, a straight portion 233b that extends from the radius portion 233a, and a mating portion 233c that extends from the straight portion 233b. A central passage 235 extends through the lower clamp portion 230. The central passage 235 has a diameter that is substantially identical to that of the upper section 215a of the central passage 215 of the upper clamp portion 210. Together, the central passage 235 of the lower clamp portion 230 and the upper section 215a of the central passage 215 of the upper clamp portion 210 form a central passage through the integrated clamp assembly 200.

The straight portion 233b of the lower clamp 230 is a generally cylindrical member that extends upward from the radius portion 233a. The external diameter of the mating portion 233c is smaller than the external diameter of the straight portion 233b, and is a light press fit to the internal diameter of the lower section 215b of the central passage 215 of the upper clamp portion 210. Thus, the mating portion 233c is adapted to match the bottom portion 213 of the upper clamp 210 to form the sliding diameter of the clamp assembly 200 when the upper and lower clamps are captured on assembly in the large slots in any connecting beam.

The lower clamp portion 230 includes a seat 240 in the form of a recess formed on the bottom of the bottom portion 232 of the lower clamp. The seat 240 is adapted to receive and engage the shoulder 127 of an anchor screw 120 (FIG. 8A-D) in a manner that allows the swing bolt 140 and the clamp assembly 200 to rotate and pivot relative to one another. This ability of the clamp assembly 200, the anchor screw 120, and the swing bolt 140 to rotate and pivot relative to one another is a feature that facilitates the selective axis feature of the spinal fixation system 100.

The shape of the recess forming the seat 240 may be selected to obtain a desired performance characteristic of the anchor screw assembly 110. For example, in a preferred embodiment illustrated in FIGS. 12A-C, the seat 240 takes the form of a spherical recess that is concentric with the central passage 235 in the lower clamp portion 230. In this embodiment, the wall 241 of the seat 240 has a curvature defined by a spherical radius, and is therefore adapted to rotatably engage a spherical shoulder 127 of a suitable anchor screw 120.

Turning to FIGS. 13A-C, in this alternative embodiment, the seat 240 is in the form of a conical or tapered recess that is concentric with the central passage 235. In this embodiment, the wall 241 of the seat 240 is flat, rather than spherical, and is inclined at an angle “beta” (“β”), relative to the plane of the disc-shaped bottom portion 232. In the preferred embodiment, the angle “beta” (“β”) is approximately 45°.

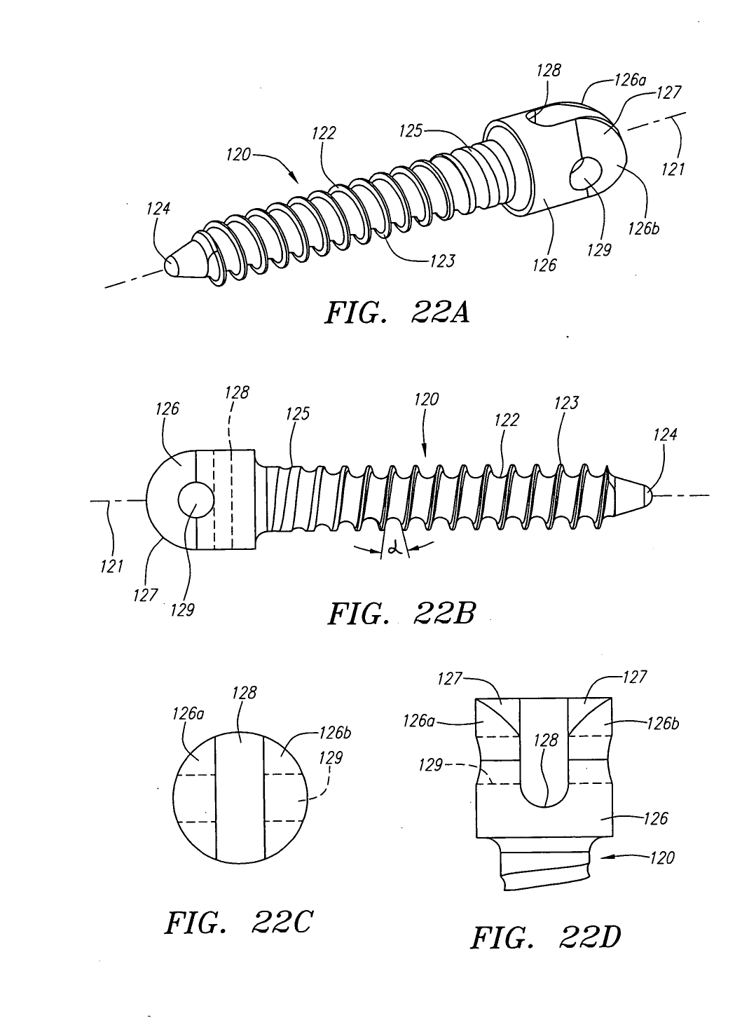

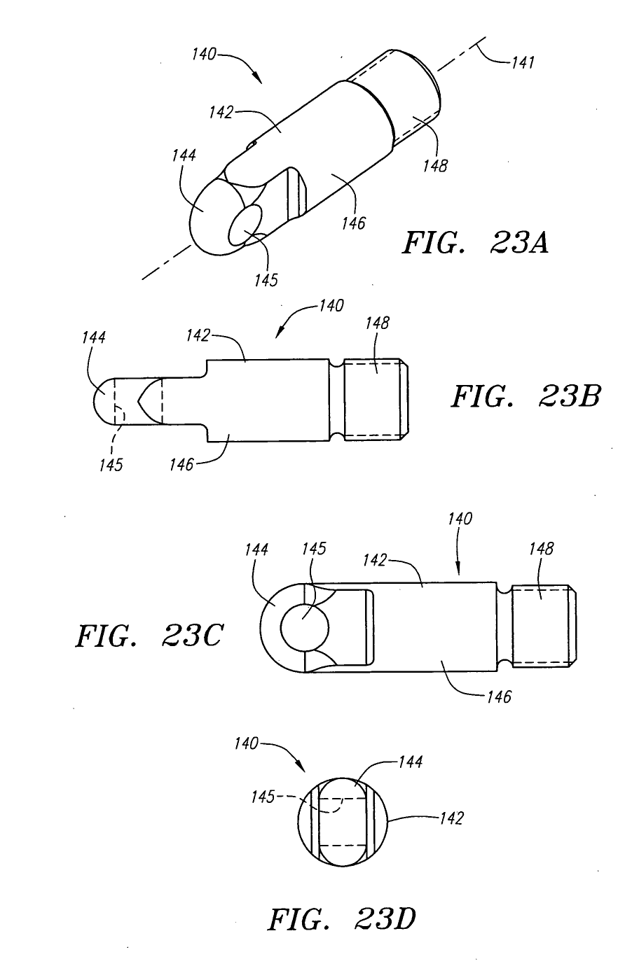

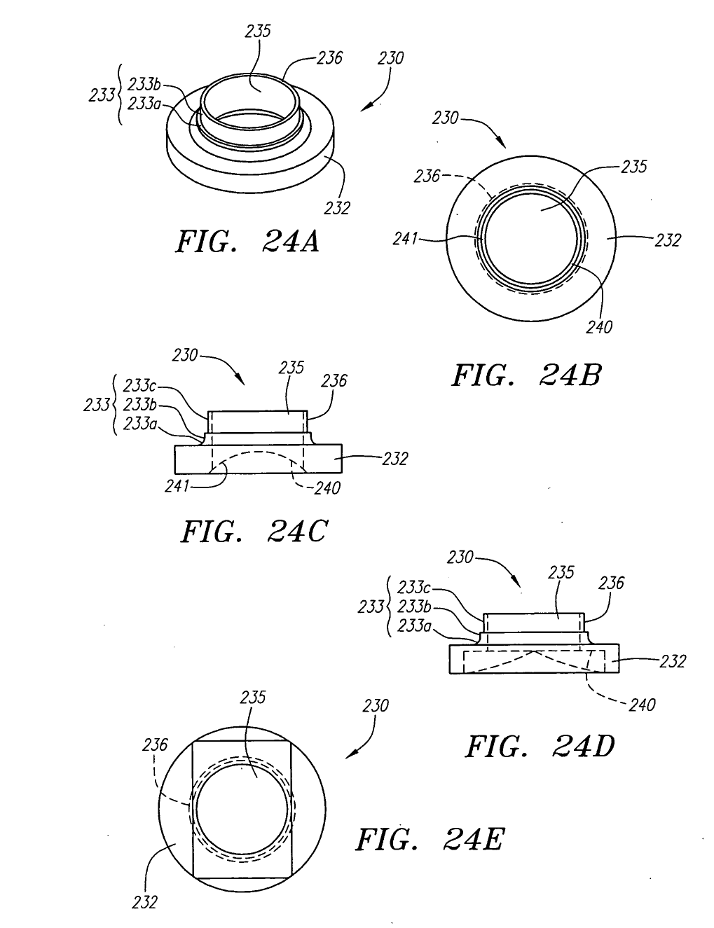

Turning to FIGS. 24A-E, in this further alternative embodiment, the seat 240 is in the form of a cylindrical recess that is concentric with the central passage 235. With particular reference to FIG. 24D, in this embodiment, the wall 241 of the seat 240 takes the form of a cylindrical cutout from the bottom of the bottom portion 232. The cylindrical seat is adapted to engage an anchor screw 120 having a cylindrical shoulder 127, as shown, for example, in FIGS. 22A-B.

Turning to FIGS. 14A-C, the one-level connecting beam 300 may include a generally flat, elongated member 310 having a rounded portion 314 at each end. Each end of the elongated member 310 also includes an elongated slot 311a-b, which slots are elongated in the direction of the longitudinal axis 315 of the elongated member 310. As noted above, the width of each of the elongated slots 311a-b is slightly larger than the external diameter of the waist portion of the clamp assembly 200 to be used with the connecting beam. The lengths of the elongated slots 311a-b are not critical, but may be long enough to provide sufficient adjustment of the position of the clamp assembly 200 within the slot 311a-b, while not compromising the strength or integrity of the elongate member 310. As noted previously, a central aperture 312 may be provided on the elongate member 310.

It is advantageous to provide rounded edges on all of the edge surfaces of the connecting beam 300, in order to minimize tissue irritation or stress risers, e.g. of tissue overlying the connecting beam 300 after implantation of the system 100. These rounded edges are illustrated in the drawings as, for example, at reference numeral 316 on the connecting beam 300 shown in FIGS. 14A-C.

Turning to FIGS. 18A-C, the two-level connecting beam 400 may include a generally flat, elongated member 410 having a rounded portion 414 at each end. An elongated slot 411a-c is provided at each end and in the center of the elongated member 410, which slots are elongated in the direction of the longitudinal axis 415 of the elongated member 410. As noted above, the width of each of the elongated slots 411a-c is slightly larger than the external diameter of the waist portion of the clamp assembly 200 to be used with the connecting beam. The lengths of the elongated slots 411a-c are not critical, but may be long enough to provide sufficient adjustment of the position of the clamp assembly 200 within the slot 411a-c, while not compromising the strength or integrity of the elongate member 410. As noted previously, one or more central apertures 412a-b may be provided on the elongate member 410.

It is advantageous to provide rounded edges on all of the edge surfaces of the connecting beam 400, in order to minimize tissue irritation or stress risers, e.g. of tissue overlying the connecting beam 400 after implantation of the system 100. These rounded edges are illustrated in the drawings as, for example, at reference numeral 416 on the connecting beam 400 shown in FIGS. 18A-C.

Turning next to FIGS. 25-28 and 30-31, the pre-formed connecting beam may include a pair of end segments 720a-b and a center segment 740. Each of the end segments 720a-b is integrally formed with the center segment 740. Each end segment extends in a plane that forms a connecting angle, typically an acute angle, with the plane in which the center segment extends. For example (see FIG. 26), the plane of the first end segment 720a forms a first connecting angle Ω with the plane of the center segment 740, and the plane of the second end segment 720b forms a second connecting angle Δ with the plane of the center segment 740. The first and second connecting angles Ω and Δ may be the same, or they may be different, but each is independently selected for any given anatomical use. Preferably, each of the connecting angles falls within the range of from about 0° to about 25°, depending upon the clinical need.

Each of the end segments 720a-b is a generally flat, plate-like member that extends away from the center segment 740. As shown in FIG. 26, each end segment 720a-b includes an elongated slot 721a-b formed near the rounded end 724a-b of the end segment. Each of the slots 721a-b is elongated in the direction of an axis extending from the center segment to the ends of each of the end segments. As noted above, the width of each of the elongated slots 721a-b is slightly larger than the external diameter of the waist portion of the clamp assembly 200 to be used with the connecting beam. The lengths of the elongated slots 721a-b are not critical, but may be long enough to provide sufficient adjustment of the position of the clamp assembly 200 within the slot 721a-b, while not compromising the strength or integrity of the connecting beam. As noted previously, one or more central apertures 722a-b may be provided on the connecting beam 700.

The center segment 740 may have a relatively shorter length than the length of either of the end segments 720a-b. The center segment 740 may also include an elongated slot 741, although the elongated slot 741 of the center segment is preferably elongated in a direction transverse to the longitudinal axis of the end segments, i.e., offset by 90 degrees relative to the longitudinal slots 721a-b on the end segments. The offset of the center segment slot 741 provides an additional degree of adjustability for the spinal fixation system.

Alternatively, the center segment 740 may be provided with no elongated slot, as shown, for example, in FIG. 27. In such a case, the center segment 740 is a generally flat segment interconnecting the pair of end segments 720a-b. In still further alternative structures of the pre-formed connecting beam, the center segment 740 may be removed altogether, thereby having a pair of end segments connected directly to one another and defining a connecting angle therebetween.

As noted above, it is advantageous to provide rounded edges on all of the edge surfaces of the connecting beam 700, in order to minimize tissue irritation or stress risers, e.g. of tissue overlying the connecting beam 700 after implantation of the system 100.

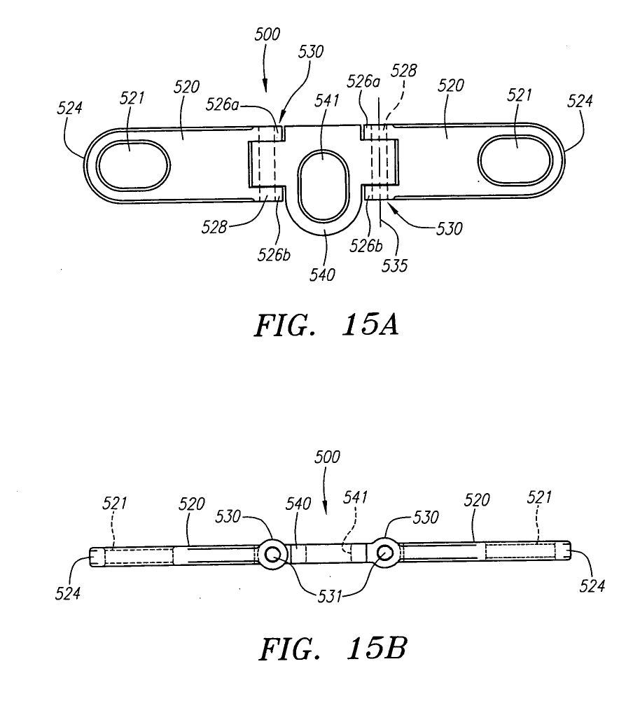

Turning next to FIGS. 15A-B, 16A-C, and 17A-C, the multi-level hinged connecting beam may include a pair of side-portions 520 and a center-portion 540. Each of the side-portions 520 is connected to the center portion 540 by a hinge 530 and hinge pin 531, thereby allowing each of the side portions 520 to pivot around the axis 535 defined by its respective hinge pin 531. Each side portion 520 has a rounded end 524, similar to the rounded ends found on the one-level and two-level connecting beams described above.

Each side portion 520 is provided with an elongated slot 521 near the rounded end 524 opposite the hinge 530. The elongated slots 521 are elongated in the direction of the longitudinal axis 525 of the side-portion 520. As noted above, the width of each of the elongated slots 521 is slightly larger than the external diameter of the waist portion of the clamp assembly 200 to be used with the connecting beam. The lengths of the elongated slots 521 are not critical, but may be long enough to provide sufficient adjustment of the position of the clamp assembly 200 within the slot 521, while not compromising the strength or integrity of the side-portion 520.

The center portion 540 also includes an elongated slot 541, although the elongated slot 541 of the center-portion is preferably elongated in a direction transverse to the longitudinal axis 525 of the side-portions, i.e., offset by 90 degrees relative to the longitudinal slots 521 on the side-portions. As described below, the offset of the center-portion slot 541 provides an additional degree of adjustability for the spinal fixation system.

With reference particularly to FIGS. 17A-C, on each side portion 520 an opposed pair of short cylindrical hinge members 526a-b are formed on the end opposite the rounded end 524. The cylindrical members 526a-b are aligned along the hinge pin axis 535, and are separated by a space 527. Each of the cylindrical members 526a-b is provided with a cylindrical passage through its center, the passage having a diameter sized to accommodate the hinge pin 531. The cylindrical members 526a-b form part of the hinge 530 (FIG. 15B) connecting the side-portions 520 to the center-portion 540.

With reference particularly to FIGS. 16A-C, the center-portion 540 includes a single short cylindrical member 546a-b formed on each of two sides of the center-portion 540. The length of the short cylindrical members 546a-b is generally about the same as the length of the space 527 separating the pairs of cylindrical members 526a-b contained on each of the side-portions 520. The cylindrical members 546a-b on the center-portion each include a passage 548 therethrough, which passages 548 are also sized to receive and retain the hinge pin 531.

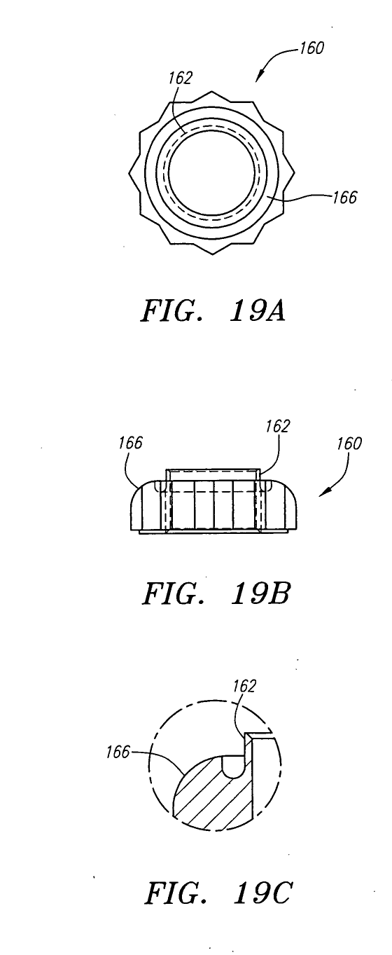

Turning to FIGS. 19A-C, a fastener, e.g., a twelve-point jam nut 160, may be used to attach the clamp assembly 200 (FIG. 7) to the connecting beams 300, 400, 500, 700 and the anchor screw assembly 110. The jam nut 160 preferably has rounded edges 166, which may minimize tissue irritation, e.g., of tissue overlying the nuts 160 after implantation of the system 100. In addition, the jam nuts 160 may include a crimpable rim 162, which may be crimped when the nuts are tightened to a desired torque, e.g., to prevent subsequent loosening of the nuts. Alternatively, hex nuts or other fasteners may be used.

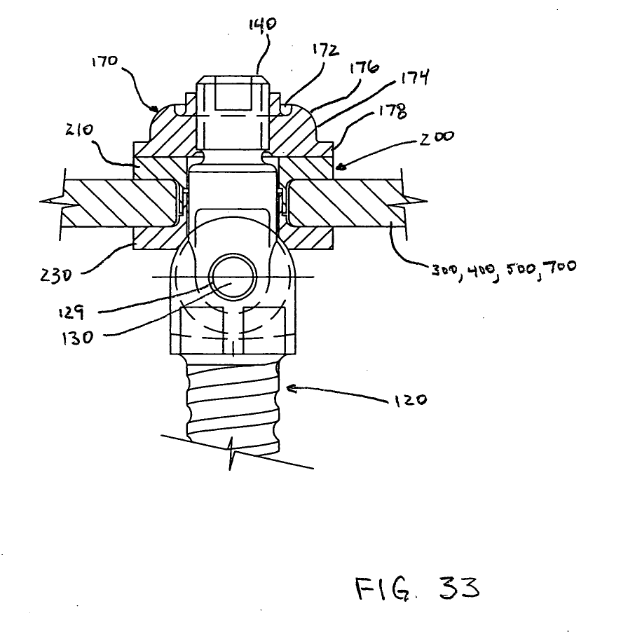

An alternative fastener is shown in FIGS. 32A-B. The fastener, a hex nut 170, is provided with rounded edges 176 for minimizing tissue irritation, and also has a crimpable rim 172, as described above. The hex nut 170 is also provided with an enlarged flange 178 extending on the bottom portion of the hex nut 170. The enlarged flange 178 extends below the gripping surfaces 174 of the hex nut, forming the bottom surface of the fastener. The enlarged flange 178 forms the surface that meets, for example, a facing surface of the clamp assembly 200 (see FIG. 33) when the spinal fixation system is assembled. The provision of an enlarged flange 178 allows the fastening force applied by the fastener to be applied over a broader surface area relative to a similar fastener that does not include the enlarged flange 178. This provides a more secure mechanism for fastening a connecting beam 300, 400, 500, 700 to an anchor screw assembly 110.

FIGS. 7, 20A, 20B, and 33 provide illustrations showing additional details of the manner in which the clamping assembly 200 engages the connecting beam. As shown in those drawings, the head portion 126 of an anchor screw 120 includes a shoulder 127 that engages the seat formed on the bottom surface of the lower clamp portion 230. Where the shoulder 127 and seat 240 are both spherical, the clamp assembly 200 is able to freely rotate and pivot relative to the shoulder 127. Alternative shoulder 127 and seat 240 combinations are also possible, such as conical or cylindrical shapes for each.

The swing bolt 140 is attached to the head portion 126 of the anchor screw 120 by a pin 130 that extends through a pin hole 129 on the anchor screw 120. The swing bolt 140 extends up through the central passage in the clamp assembly 200, where the jam nut 160, 170 is attached to it.

The clamp assembly 200 includes the upper clamp portion 210 and the lower clamp portion 230. The bottom portion 213 of the upper clamp 210 and the mating portion 233c of the lower clamp are engaged to form a narrowed waist 250 of the clamp assembly. The narrowed waist 250 is of a diameter that allows it to slidably engage the elongated slot of the connecting beams 300, 400, 500, 700 whereas the disc-shaped top portion 212 of the upper clamp 210 and the disc-shaped bottom portion 232 of the lower clamp are adapted to engage the surfaces of the connecting plate 300 when the jam nut 160, 170 is tightened. (Note: Although the one-level connecting beam 300 is shown in FIGS. 7, 20A, and 20B, the foregoing descriptions apply equally to the other connecting beams described herein).

As described previously herein, the bottom surface of the jam nut 160, 170 engages the upper surface of the upper clamp 210 to provide the fastening force for connecting the connecting beam to the anchor screw assembly. A comparison of the structures shown in FIGS. 7, 20A, 20B with the structure shown in FIG. 33 shows the increased amount of surface area available for providing the fastening force due to the provision of the flange 178 on the bottom surface of the jam nut 170, and the provision of an increased diameter upper clamp 210. The flange 178 shown in the jam nut 170 embodiment illustrated in FIG. 33 has a diameter that is about 28% larger than the effective diameter of the jam nut 160. Flanges having a greater or lesser diameter may also be possible. The upper clamp 210 preferably has the same dimension as the flange 178.

Turning to FIGS. 27-31, the spinal fixation systems described herein may also include one or more cross-brace members 800 extending between and interconnecting two or more connecting beams. The preferred cross-brace member comprises a flat, elongated member 810, each end of which is attached to a separate connecting beam. The flat, elongated member 810 is preferably formed of the same materials used to make the connecting beams 300, 400, 500, 700, described elsewhere herein. Additionally, the flat, elongated member 810 preferably has cross-sectional dimensions (e.g., width, plate thickness) the same as or similar to the connecting beams. However, because it performs different functions and bears different types of loads, the flat, elongated member 810 may also have very different dimensions than those of the connecting beams.

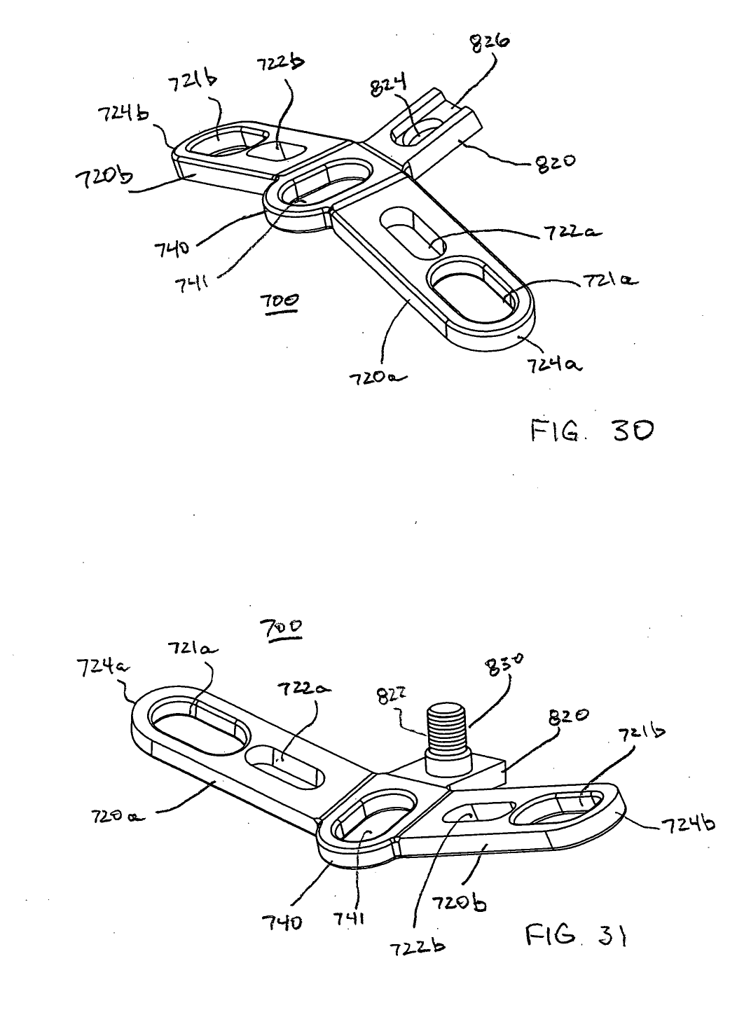

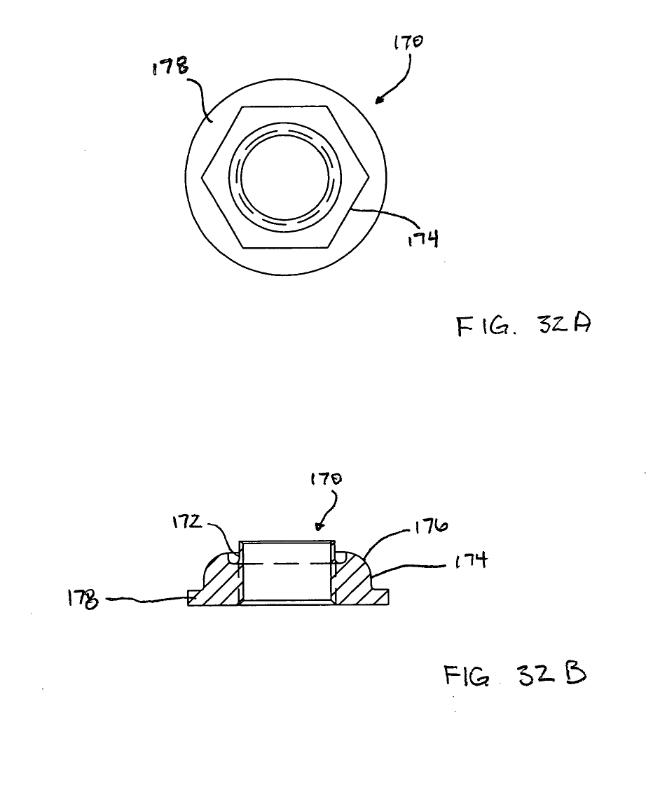

The cross-brace member 800 may be connected to its associated connecting beams by any suitable mechanism. Preferably, each connecting beam 300, 400, 500, 700 to which the cross-brace member is to be attached is provided with an attachment base 820, which is preferably an integrally formed member that extends laterally a short distance from one side of the connecting beam 300, 400, 500, 700. (See, e.g., FIGS. 30-31). The attachment base 820 includes a surface or structural support upon which a connecting member 822 may be assembled. The connecting member 822 is some structural member or other member that is adapted to engage or mate with a corresponding member provided on the cross-brace member 800. One example of a connecting member 822 is shown in FIG. 31. In FIG. 30, the connecting member comprises a round hole 824 formed through the attachment base 820. An anti rotation slot 826 is also formed on the attachment base 820 in an overlapping manner with the hole 824. The hole 824 and recess 826 are preferably of a size and shape to provide passage of a stud or other connecting member provided on a cross-brace member 800, or to provide passage of a bolt through both the hole 824 on the attachment base 820 and another slot formed on the cross-brace member 800. One or more fasteners, such as a jam nut 160, 170, may be attached to the stud or bolt to connect the cross-brace member 800 to the connecting beam 300, 400, 500, 700.

In FIG. 31, the connecting member 822 comprises a threaded stud 830 extending upward from the upper surface of the attachment base 820. The stud 830 has a length and diameter suitable for extending through a slot provided on an end of the cross-brace member 800. A fastener, such as a jam nut 160, 170, may be attached to the stud 830 to connect the cross-brace member 800 to the connecting beam 300, 400, 500, 700. Examples of this type of connection mechanism are shown in FIGS. 27-29.

The cross-brace members 800 described herein provide the ability to obtain an additional amount of structural integrity to the spinal fixation systems, and to obtain relative fixation between two (or more) spinal bodies in a manner that does not require implantation of additional anchor screw assemblies. The cross-brace members 800 may be used with any of the connecting beam structures described herein, such as the one-level connecting beams 300 (see FIG. 29), two-level connecting beams 400, multi-level connecting beams 500, the pre-formed connecting beams 700 (see FIGS. 27-28), or any combinations of any of these connecting beams.

To provide a system for treating vertebrae of a patient, a set of anchor screws, clamp assemblies, fasteners, and one or more connecting beams may be selected based upon the specific vertebrae being treated and/or based upon the anatomy encountered. Cross-brace members may optionally be included as well. A system in accordance with the present invention provides a modularity that may easily accommodate a variety of anatomy in patients.

Turning again to FIGS. 1 and 2, exemplary spinal fixation systems 100 are shown implanted along a spinal column, each of which includes three anchor screw assemblies 110, three clamp assemblies 200, three fasteners 160, and a multi-level connecting beam 500 per side. In the alternative, fewer or additional anchor screws may be implanted and other types or lengths of connecting beams may be used, e.g., to fix fewer or additional vertebrae.

Preferably, each of the spinal fixation systems 100 is implanted generally parallel to the central spinal axis on either side of the spinous processes 602, as shown in FIG. 1. The system 100 may be used to provide adjustment of the vertebrae, e.g., to allow vertical or horizontal, medial or lateral adjustment. Although an implantation procedure for only one assembly 100 is described below, it will be appreciated that a second assembly (or even additional assemblies) may be implanted using a similar procedure.

Turning first to FIGS. 1 and 2, the vertebrae, e.g., vertebrae 610, 620, 630, to be stabilized are exposed, e.g., using conventional surgical procedures. The anchor screws 120 are screwed into the vertebrae 610-630, respectively, e.g., into the pedicles, generally in a substantially straight line (except at the vertebra 620). Preferably, the anchor screws 120 are screwed in sufficiently to provide a predetermined pivot axis with respect to a centerline spinal axis of the patient. For example, the anchor screws 120 that are screwed into the first and third vertebra 610, 630 shown in FIG. 1, may be screwed until the pivot axes of the anchor screws 120 are disposed generally parallel to the centerline spinal axis. In contrast, the second anchor screw 120, which is screwed into the second vertebrae 620, may be screwed in until the pivot axis is disposed substantially transverse to the first and third pivot axes, and preferably substantially perpendicular to the centerline spinal axis.

Next, the clamp assemblies 200, assembled onto the connecting beams, 300, 400, 500, or 700 are placed over the swing bolts 140 which are attached to the anchor screws 120 that have been screwed into the adjacent vertebrae 610, 620, 630. Several different embodiments of the clamp assemblies 200 and connecting beams 300, 400, 500, 700 are described herein, any of which may be selected for a particular application, though it is preferred to select a clamp assembly 200 having a spherical seat 240 that is adapted to engage the spherical shoulder 127 of its respective anchor screw 120 in a manner that provides desired results. The clamp assemblies 200 may be received over the intermediate regions 146 of the respective swing bolts 140, until the seats 240 engage the anchor screw shoulders 127.

The connecting beam 300, 400, 500, 700 with clamp assemblies 200 in position may then be received over the intermediate regions 146 of the swing bolts 140 on assembly. More particularly, each swing bolt 140 is extended through one of the clamp assemblies 200 assembled into the elongated slots 521 of the connecting beam 500, with the narrowed waist portions 250 of the clamp assemblies 200 each sliding in its respective elongated slot 521 as well. Once the connecting beam is properly located, and the clamp assemblies 200 are moved into position over the intermediate regions 146 of the swing bolts 140, jam nuts 160, 170 are assembled onto the threaded regions 148 of the swing bolts 140 and the subsequent assembly is tightened to the prescribed torque.

The selective axis feature of the spinal fixation system 100 may be obtained with the system thus described. On assembly, the spherical shoulder 127 of the anchor screw 120 loads against the spherical seat 240 of the lower clamp 230. The swing bolt 140 of the anchor screw assembly 110 passes up through the central passage formed by the clamp assembly 200. The jam nut 160, 170 is threaded onto the end of the swing bolt 140 down to the flat upper face of the upper clamp 210. On final assembly, the jam nut 160, 170 and the spherical shoulder 127 of the anchor screw 120 tighten the clamp assembly 200 firmly against the connecting beam 300, 400, 500, 700 at 90° force. Prior to final tightening of the jam nut 160, 170, the clamp assembly 200 slides in the connecting beam slot 521 to locate the swing bolt 140 centerline, after which the jam nut 160, 170 is tightened at approximately 120-140 inch pounds of force.

Optionally, one or more cross-brace members 800 may be provided to extend between and interconnect two or more connecting beams 300, 400, 500, 700. Preferably, the connecting beams are each provided with an attachment base 820 having a connecting member 822 (such as a threaded stud 830) formed thereon. The cross-brace members 800 are then attached to each of the connecting beams using a fastener, such as a jam nut 160, 170.

By way of example, the present application describes selective axis posterior lumbar spinal plating fixation apparatuses and methods for their use. It should be understood, however, that the apparatuses and methods described may be readily adapted for use in other applications, such as for a posterior cervical plating application.

While the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents and alternatives falling within the spirit and scope of the appended claims.

1. A spinal fixation system, comprising:
a first anchor screw assembly comprising a first screw having a threaded portion, a first swing bolt pivotally coupled to the screw and comprising an intermediate region extending along a longitudinal axis of the swing bolt, a first clamp assembly comprising a first passage for receiving the first swing bolt therethrough, the intermediate region and the first passage having similar cross-sections, and a first fastener for securing the first clamp assembly on the first swing bolt;
a second anchor screw assembly comprising a second screw having a threaded portion, a second swing bolt pivotally coupled to the second screw and comprising an intermediate region extending along a longitudinal axis of the second swing bolt, a second clamp assembly comprising a second passage for receiving the second swing bolt therethrough, the intermediate region and the second passage having similar cross-sections, and a second fastener for securing the second clamp assembly on the swing bolt; and
a connecting member having a plurality of slots extending therethrough, wherein each of the first and second clamp assemblies are adapted to slidably engage one of said slots, and wherein said connecting member comprises a first segment extending in a first plane, and a second segment extending in a second plane, said first plane and said second plane defining a first connecting angle therebetween.

2. The spinal fixation system of claim 1, wherein the first swing bolt comprises a threaded region opposite the first screw, and wherein the first fastener comprises a nut threadable onto the threaded region of the first swing bolt.

3. The spinal fixation system of claim 2, wherein the intermediate region of the first swing bolt comprises a smooth walled region for slidably receiving the first clamp assembly thereon, the smooth walled region being located between the threaded region and the first screw.

4. The spinal fixation system of claim 1, wherein the first clamp assembly comprises first and second clamp portions, each clamp portion having a first passage therethrough for receiving the first swing bolt therethrough, the first clamp assembly further comprising a narrowed waist portion for engaging a slot of said connecting member.

5. The spinal fixation system of claim 1, wherein the first clamp assembly comprises:
an upper clamp portion having a generally disc-shaped top portion and a smaller generally cylindrical bottom portion, and having a generally cylindrical central passage therethrough, the central passage having an upper section and a lower section wherein the upper section has a predetermined size for fitting an intermediate region of a swing bolt, and the lower section has an opening slightly larger than that of the upper section for mating with a lower clamp portion to provide a press fit therewith, and
a lower clamp portion having a generally disc-shaped bottom portion and a generally cylindrical shaped top portion, and a mating portion extending from an intermediate straight portion for forming a light press fit with the inside of the lower section of the central passage of the upper clamp portion, and adapted to form a sliding fit of the clamp assembly within an opening in a connecting beam, and the lower clamp portion includes a central passage therethrough having a size substantially identical to that of the upper section of the central passage of the upper clamp portion for receiving therethrough a swing bolt, and the lower clamp portion having a curved seat in the form of a recess formed in the bottom of the bottom portion of the lower clamp portion adapted to receive and engage a curved shoulder of an anchor screw in a manner to allow a swing bolt and the resulting clamp assembly to rotate and pivot relative to one another to facilitate selective axis spinal fixation.

6. A spinal fixation system as in claim 5 wherein the seat of the lower clamp portion is spherical.

7. A spinal fixation system as in claim 5 wherein the seat of the lower clamp portion is conical.

8. A spinal fixation system as in claim 5 wherein the seat of the lower clamp portion is cylindrical.

9. A spinal fixation system as in claim 5 wherein a lower surface of the generally disc-shaped top portion of the upper clamp portion and the generally disc-shaped bottom portion of the lower clamp portion are adapted to firmly clamp onto a connecting beam as a result of tightening a fastener onto an end of a swing bolt extending the central passages of the upper and lower clamp portions.

10. A spinal fixation system as in claim 5 including a plurality of upper clamp portions and lower clamp portions for receiving a like plurality of swing bolts and for forming a fit for each resulting clamp assembly with different openings in a connecting beam.

11. A spinal fixation system as in claim 5 wherein the mating portion extending from the intermediate straight portion of the lower clamp portion has an external diameter smaller than the internal diameter of the opening in the lower section of the upper clamp portion to provide a light press fit when the upper and lower clamp portions are captured in an opening in a connecting beam.

12. The spinal fixation system of claim 1, wherein the first screw comprises a shoulder adjacent the first swing bolt, and wherein the first clamp assembly substantially engages the shoulder when the first clamp assembly is secured on the first swing bolt in a manner that allows the first swing bolt to pivot with respect to the first screw.

13. The spinal fixation system of claim 1, wherein said connecting member comprises:
a first segment having a first slot formed therein, and
a second segment having a second slot formed therein.

14. The spinal fixation system of claim 13, further comprising:
a third segment extending in a third plane and having a third slot formed therein, said third segment located between and separating said first segment and said second segment, and
wherein said first segment and said third segment define a second connecting angle therebetween, and said second segment and said third segment define a third connecting angle therebetween.

15. A method for stabilizing vertebrae relative to one another using a plurality of swing bolt anchor screws, each swing bolt anchor screw comprising a swing bolt pivotally coupled to a threaded portion, the vertebrae being disposed adjacent one another along a central spinal axis, the method comprising:
screwing a threaded portion of a first swing bolt anchor screw into a first vertebra until a first pivot axis of the first swing bolt anchor screw has a predetermined orientation with respect to the spinal axis;
screwing a threaded portion of a second swing bolt anchor screw into a second vertebra adjacent the first vertebra until a second pivot axis of the second swing bolt anchor screw is substantially transverse to the first pivot axis;
placing a connecting member on the swing bolts of the first and second swing bolt anchor screws, the connecting member having at least one clamp member engaged in at least one slot through which at least one of the swing bolts extends, the connecting member extending at least between the first and second anchor screws and having at least a first segment in a first plane and a second segment in a second plane, with said first plane and said second plane defining a connecting angle therebetween; and
securing the at least one clamp member on at least one of the swing bolts of the first and second swing bolt anchor screws, thereby securing the connecting member to the at least one of the swing bolt anchor screws.

16. The method of claim 15, further comprising:
screwing a threaded portion of a third anchor screw into a third vertebra adjacent the first vertebra;
placing the connecting member on a swing bolt of the third anchor screw, the connecting member thereby also extending between the first swing bolt anchor screw and the third anchor screw; and
securing the connecting member on the third anchor screw.

17. The method of claim 16, wherein the third anchor screw comprises a third swing bolt anchor screw, wherein the threaded portion of the third swing bolt anchor screw is screwed into the third vertebra until a third pivot axis of the third swing bolt anchor screw is substantially transverse to the second pivot axis, and wherein a clamp member engaged in a slot on the connecting member is placed on a swing bolt of the third swing bolt anchor screw.

18. The method of claim 15, wherein the threaded portions of the first and second swing bolt anchor screws comprise head portions including shoulders, the swing bolts being pivotally coupled to the head portions, and wherein a lower portion of the clamp member frictionally engages at least one of the shoulders, thereby securing the at least one of the swing bolts with respect to the threaded portions.

19. The method of claim 15, wherein the swing bolts comprise threaded regions, and wherein the clamp members are secured on the swing bolts by threading a fastener onto the threaded regions.

20. The method of claim 15, wherein the predetermined orientation of the first pivot axis is generally parallel to the spinal axis.

21. A spinal fixation system, comprising:
a first pair of anchor screws, each of which is connected to a first connecting beam;
a second pair of anchor screws, each of which is connected to a second connecting beam;
a brace member interconnecting said first connecting beam and said second connecting beam.

22. The spinal fixation system of claim 21, wherein said first connecting beam comprises an attachment base to which the brace member is connected.

23. The spinal fixation system of claim 22, further comprising a stud formed on a surface of said attachment base, the stud adapted to engage a slot formed on said brace member.

24. The spinal fixation system of claim 22, further comprising a bolt extending through a first slot formed on said attachment base and through a second slot formed on said brace member.

25. The spinal fixation system of claim 21, wherein said first connecting beam comprises a first segment extending in a first plane, and a second segment extending in a second plane, said first plane and said second plane defining a first connecting angle therebetween.

26. The spinal fixation system of claim 21, wherein said first connecting beam comprises:
a first connecting member section having a first slot formed therein,
a second connecting member section having a second slot formed therein, and
a first hinge connecting said first and second connecting member sections.

27. The spinal fixation system of claim 26, wherein said first connecting beam further comprises:
a third connecting member section having a third slot formed therein, and
a second hinge connecting said second and third connecting member sections.
